(19) United States
(12) Patent Application Publication
(10) Pub. No.: US 2021/0348231 A1
(43) Pub. Date: Nov. 11, 2021

(54) DETECTION OF DRUG-RESISTANT MYCOPLASMA GENITALIUM

(71) Applicant: GEN-PROBE INCORPORATED, San Diego, CA (US)

(72) Inventors: Tamara JOHNSON, San Diego, CA (US); Paul M. DARBY, San Diego, CA (US); Damon K. GETMAN, Poway, CA (US)

(21) Appl. No.: 17/382,568

(22) Filed: Jul. 22, 2021

Related U.S. Application Data
(63) Continuation-in-part of application No. PCT/US20/14810, filed on Jan. 23, 2020.
(60) Provisional application No. 62/797,053, filed on Jan. 25, 2019.

(51) Int. Cl. C12Q 1/6876; C12Q 1/6806; C12Q 1/6827

(52) U.S. Cl. CPC C12Q 1/6876; C12Q 1/6806; C12Q 2600/106; C12Q 2600/156; C12Q 1/6827

(57) ABSTRACT

Provided are methods, compositions and systems for detecting the presence or absence of nucleic acid targets, such as nucleic acids of macrolide-resistant Mycoplasma genitalium. In one embodiment, real-time Ct values determined for a wild-type sequence and for a drug resistance marker, each on an opposite strand of the same amplification product, are compared to determine the presence or absence of the drug resistance marker in nucleic acids of a test sample.

### CROSS-REFERENCE TO RELATED APPLICATION

This application is a continuation-in-part of International Application No. PCT/US20/14810, filed Jan. 23, 2020, which claims the benefit of U.S. Provisional Application No. 62/797,053, filed Jan. 25, 2019. The contents of each of these applications is incorporated by reference herein.

### TECHNICAL FIELD

The disclosure relates generally to the field of biotechnology. More specifically, the disclosure relates to compositions, methods, kits, and systems that detect macrolide-resistant Mycoplasma genitalium.

### BACKGROUND

Mycoplasmas are small prokaryotic organisms (0.2 to 0.3 μm) belonging to the class Mollicutes, whose members lack a cell wall and have a small genome size. The mollicutes include at least 100 species of Mycoplasma, 13 of which are known to infect humans.

One Mycoplasma species of clinical relevance is M. genitalium. This organism, which is thought to be a cause of nongonococcal urethritis (NGU), a sexually transmitted disease, has been detected to a significantly greater extent in symptomatic males than in asymptomatic males. See Yoshida et al., “Phylogeny-Based Rapid Identification of Mycoplasma and Ureaplasmas from Urethritis Patients,” J. Clin. Microbiol., 40:105-110 (2002). In addition to NGU, M. genitalium is thought to be involved in pelvic inflammatory disease (which can lead to infertility in women in severe cases), adverse birth outcomes, and increased risk for human immunodeficiency virus (HIV) infection. See Maniloff et al., Mycoplasmas: Molecular Biology and Pathogenesis 417 (ASM 1992); and Manhart et al., supplement to Contemporary OB/GYN (July 2017).

Significantly, M. genitialium is more common than many other sexually transmitted pathogens. Studies of low-risk individuals estimated the prevalence of M genitialium among women to be in the range of from 0.8%-4.1%, and among men to be in the range of from 1.1%-1.2%. Among the population of women attending an STI clinic, the prevalence of M. genitialium ranged as high as 19% in two major U.S. cities. The prevalence was as high as 15% for men attending the STI clinics. In recent studies, M. genitialium prevalence was higher than all other bacterial sexually transmitted infections.

The advent and spread of antibiotic-resistant strains of M. genitalium renders infection control more difficult. Current treatment protocols for infection with M. genitialium rely on administration of the macrolide antibiotic azithromycin. One study conducted in Australia more than a decade ago revealed evidence for progressive dissemination of M. genitialium bacteria that were resistant to this treatment. The resistance was attributed to adjacent mutations at two positions in the 23S rRNA that could be detected using nucleic acid sequencing or “high resolution melt analysis” techniques. Unfortunately, nucleic acid sequencing approaches do not lend themselves to rapid testing, and melt curve analyses, although effective, had trouble differentiating genotypes (i.e., wild-type and mutants). Benefits of early detection include the opportunity to reduce transmission of resistant M. genitialium strains in the community and shortening the time to effective second line treatment. (See Twin et al., PLoS ONE 7(4): e35593. Doi:10.1371/journal.pone.0035593)

Sensitive and highly specific molecular tests for nucleic acids of M. genitialium have been described in U.S. Pat. No. 7,345,155, the disclosure of which is incorporated by reference. However, these tests do not detect the macrolide resistance genetic marker. The present disclosure provides supplemental techniques that can be used for detecting the genetic marker of macrolide resistance in M. genitialium.

### SUMMARY OF THE DISCLOSURE

In a first aspect, the disclosure relates to a method of determining the presence or absence of a nucleic acid target sequence in a test sample. The method includes the step of (a) obtaining nucleic acid from the test sample. There also is the step of (b) performing an in vitro nucleic acid amplification reaction using a pair of primers and nucleic acid obtained in step (a) as templates to produce an amplification product having first and second nucleic acid strands that are complementary to each other, wherein the first nucleic acid strand includes a positive control sequence, and where the second nucleic acid strand may include the nucleic acid target sequence. There also is the step of (c) detecting, as the in vitro nucleic acid amplification reaction is taking place, the positive control sequence in the first nucleic acid strand and any of the nucleic acid target sequence that may be present in the second nucleic acid strand to determine Ct values for each of the positive control sequence and the nucleic acid target sequence. There also is the step of (d) comparing the determined Ct values to establish the presence or absence of the nucleic acid target sequence in the test sample. According to one generally preferred embodiment, step (c) can involve detecting with invasive cleavage reactions. In some embodiments, Ct values determined for the positive control sequence and the nucleic acid target sequence are not identical when both the positive control sequence and the nucleic acid target sequence are both present in the amplification product produced in step (b).

In a second aspect, the disclosure relates to a method of determining the macrolide resistance status of M. genitalium in a test sample. The method includes the step of (a) obtaining nucleic acid from M. genitalium of the test sample. There also is the step of (b) performing an in vitro nucleic acid amplification reaction using nucleic acid obtained in step (a) as templates to produce an amplification product including a segment of M. genitalium 23S ribosomal nucleic acid, where the segment includes two adjacent nucleotide positions, corresponding to positions 2058 and 2059 of region V in E. coli 23S rRNA, that distinguish macrolide-sensitive and macrolide-resistant M. genitalium, and where the segment further includes a wild-type sequence of M. genitalium 23S ribosomal nucleic acid. There also is the step of (c) detecting in the amplification product, as the in vitro nucleic acid amplification reaction of step (b) is occurring, the wild-type sequence, and any of a macrolide resistance marker that may be present at either of the two adjacent nucleotide positions to determine Ct values for each of the wild-type sequence and the macrolide resistance marker. There also is the step of (d) comparing the determined Ct values to establish the presence or absence of the macrolide resistance marker in the amplification product, thereby determining the macrolide resistance status of M. genitalium in the test sample. According to one generally preferred embodiment, the amplification product produced in the in vitro nucleic acid amplification reaction of step (b) includes a double-stranded DNA. In some embodiments, when the amplification product produced in the in vitro nucleic acid amplification reaction of step (b) includes a double-stranded DNA, step (c) can involve detecting the wild-type sequence and the macrolide resistance marker on different strands of the double-stranded DNA. In some embodiments, the in vitro nucleic acid amplification reaction of step (b) can include a flap endonuclease (FEN) enzyme, and step (c) can involve detecting with a plurality of invasive cleavage reactions. For example, the in vitro nucleic acid amplification reaction can be a PCR reaction employing first and second primers oriented opposite to each other, and one of the primers can be an invasive probe that promotes cleavage of a first primary probe to release a first 5′-flap oligonucleotide in the presence of the FEN enzyme. In certain preferred embodiments, the first primary probe is specific for the wild-type sequence, and is cleaved by the FEN enzyme if hybridized to any of the amplification product that includes the wild-type sequence. In some embodiments, the macrolide resistance marker is either A2058C, A2058T, or A2058G. In some embodiments, the macrolide resistance marker is A2059G. In some embodiments, when the amplification product produced in the in vitro nucleic acid amplification reaction of step (b) includes a double-stranded DNA, step (c) can involve detecting with a plurality of invasive cleavage reactions. In some embodiments, the plurality of invasive cleavage reactions distinguishes the wild-type sequence from the macrolide resistance marker, but does not distinguish any of A2059G, A2058C, A2058T or A2058G from each other. In some embodiments, when the amplification product produced in the in vitro nucleic acid amplification reaction of step (b) includes a double-stranded DNA, a set of four primary probes is used to detect the macrolide resistance marker at either of the two adjacent nucleotide positions in one strand of the double-stranded DNA, and each probe among the set shares the same 5′-flap sequence. In some embodiments, when the amplification product produced in the in vitro nucleic acid amplification reaction of step (b) includes a double-stranded DNA, a set of four primary probes can be used to detect the macrolide resistance marker at either of the two adjacent nucleotide positions in one strand of the double-stranded DNA, and step (c) can involve detecting with a single invasive probe that cleaves a 5′-flap from any of the four primary probes among the set in the presence of a complementary DNA strand including any of A2059G, A2058C, A2058T and A2058G. In some embodiments, cleavage of a single FRET cassette separates a fluorophore and a quencher following hybridization of the single FRET cassette to a 5′-flap cleaved from any primary probe among the set of four primary probes. In some embodiments, step (d) includes calculating a difference between the Ct values. In some embodiments, step (d) includes calculating a difference between the Ct values, and then determining whether the difference is greater than or less than 0 cycles. In some embodiments, the test sample includes a clinical swab sample obtained from a patient. In some embodiments, step (a) includes obtaining RNA from M. genitalium of the test sample, and step (b) involves performing the in vitro nucleic acid amplification reaction using the RNA obtained in step (a) as templates. In some embodiments, the test sample includes a mixture of macrolide-resistant M. genitalium and macrolide-sensitive M. genitalium. In some embodiments, the test sample is known to include M. genitalium prior to performing step (b), and wherein step (c) includes detecting with two different FRET cassettes, each FRET cassette being labeled with a different fluorophore. In some embodiments, step (a) includes obtaining nucleic acids by hybridization capture onto a solid support displaying immobilized oligonucleotides.

In a third aspect, the disclosure relates to an oligonucleotide composition. The composition includes a first primer complementary to a sequence of M. genitalium 23S rRNA or a DNA equivalent strand downstream of position 2059 of corresponding region V in E. coli 23S rRNA, and a second primer complementary to an extension product of the first primer using M. genitalium 23S rRNA or the DNA equivalent strand as a template, the second primer being complementary to a sequence of M. genitalium 23S ribosomal DNA upstream of position 2058 of corresponding region V in E. coli 23S rRNA. There also is a primary probe including a wild-type target-binding sequence attached to an upstream 5′-flap sequence, wherein the wild-type target-binding sequence is complementary to a wild-type sequence of M. genitalium 23S rRNA downstream of the first primer with a 1-2 base overlap at the 5′-end of the wild-type target-binding sequence when the primary probe and the first primer are hybridized to the same strand of M. genitalium 23S rRNA or the DNA equivalent strand. There also is a set of four primary probes, each probe of the set being specific for a different single nucleotide polymorphism (SNP) in M. genitalium 23S ribosomal DNA, at positions corresponding to positions 2058 and 2059 of region V in E. coli 23S rRNA, that distinguishes macrolide-sensitive and macrolide-resistant M. genitalium, wherein each of the four primary probes is specific for one of A2058C, A2058T, A2058G, and A2059G, and wherein each primary probe among the set is attached to an upstream 5′-flap sequence different from the upstream 5′-flap sequence of the primary probe including the wild-type target-binding sequence. There also is an invasive probe that promotes flap endonuclease (FEN) enzyme-mediated cleavage of a complex including the invasive probe, any of the set of four primary probes, and an M. genitalium 23S ribosomal DNA sequence from a macrolide-resistant M. genitalium but not macrolide-sensitive M. genitalium. There also are two FRET cassettes, one FRET cassette being specific for any cleaved 5′-flap released from the primary probe including the wild-type target-binding sequence, and the other FRET cassette being specific for any cleaved 5′-flap released from any of the set of four primary probes. In some embodiments, the first primer includes the sequence of SEQ ID NO:7. In some embodiments, the second primer includes the sequence of SEQ ID NO:1. In some embodiments, the primary probe including the wild-type target-binding sequence includes the target-binding sequence of SEQ ID NO:10. In some embodiments, the set of four primary probes includes a probe of the sequence SEQ ID NO:11. In some embodiments, the set of four primary probes includes a probe of the sequence SEQ ID NO:12. In some embodiments, the set of four primary probes includes a probe of the sequence SEQ ID NO:13. In some embodiments, the set of four primary probes includes a probe of the sequence SEQ ID NO:14. In some embodiments, the invasive probe includes a sequence selected from the group consisting of SEQ ID NO:8 and SEQ ID NO:9. In some embodiments, the primary probe that includes the wild-type target-binding sequence and each probe among the set of four primary probes are complementary to opposite strands of M. genitalium 23S ribosomal DNA.

In a fourth aspect, the disclosure relates to a reaction mixture. The reaction mixture includes an oligonucleotide composition in accordance with any embodiment of the above-described third aspect of the disclosure, particularly when the primary probe that includes the wild-type target-binding sequence and each probe among the set of four primary probes are complementary to opposite strands of M. genitalium 23S ribosomal DNA. There also are each of a DNA polymerase, a FEN enzyme, dNTPs, and a 23S M genitalium ribosomal nucleic acid.

### BRIEF DESCRIPTION OF THE DRAWINGS

FIG. 1 schematically illustrates features of an assay involving nucleic acid amplification (e.g., PCR) with invasive cleavage detection of amplicon synthesis as the amplification reaction is occurring.

FIGS. 2A and 2B present run curves obtained using PCR amplification with real-time invasive cleavage detection, where the invasive probe that cleaves primary probes specific for macrolide resistance markers was varied. FIG. 2A presents results obtained using the invasive probe of SEQ ID NO:8. FIG. 2B presents results obtained using the invasive probe of SEQ ID NO:9. Each panel shows results from real-time amplification and detection of 10⁶ copies of wild-type template, and 10⁵ or 10⁶ copies of the template including a drug resistance marker (A2059G).

### Definitions

Before describing the present teachings in detail, it is to be understood that the disclosure is not limited to specific compositions or process steps, as such may vary. It should be noted that, as used in this specification and the appended claims, the singular form “a,” “an,” and “the” include plural references, and expressions such as “one or more” include singular references unless the context clearly dictates otherwise. Thus, for example, reference to “an oligonucleotide” includes a plurality of oligonucleotides and the like; in a further example, a statement that “one or more secondary detection oligonucleotides are FRET cassettes” includes a situation in which there is exactly one secondary detection oligonucleotide and it is a FRET cassette. The conjunction “or” is to be interpreted in the inclusive sense (i.e., as equivalent to “and/or”), unless the inclusive sense would be unreasonable in the context. When “at least one” member of a class (e.g., oligonucleotide) is present, reference to “the” member (e.g., oligonucleotide) refers to the present member (if only one) or at least one of the members (e.g., oligonucleotides) present (if more than one).

As used herein, the term “sample” refers to a specimen that may contain macrolide-resistant M. genitialium or components thereof (e.g., nucleic acids). Samples may be from any source, such as biological specimens or environmental sources. Biological specimens include any tissue or material derived from a living or dead organism. Examples of biological samples include vaginal swab samples, respiratory tissue, exudates (e.g., bronchoalveolar lavage), biopsy, sputum, peripheral blood, plasma, serum, lymph node, gastrointestinal tissue, feces, urine, or other fluids, tissues or materials. Samples may be processed specimens or materials, such as obtained from treating a sample by using filtration, centrifugation, sedimentation, or adherence to a medium, such as matrix or support. Other processing of samples may include treatments to physically or mechanically disrupt tissue, cellular aggregates, or cells to release intracellular components that include nucleic acids into a solution which may contain other components, such as enzymes, buffers, salts, detergents and the like. Samples being tested for the presence of an analyte may sometimes be referred to as “test samples.”

As used herein, an “invasive cleavage assay” is a procedure that detects or quantifies a target nucleic acid by enzymatic cleavage of two different invasive cleavage structures. Reagents for an invasive cleavage assay include: a structure-specific 5′ nuclease; and three oligonucleotides (an “invasive probe,” a “primary probe,” and a “FRET cassette”). The invasive cleavage assay combines two invasive signal amplification reactions (i.e., a “primary reaction” and a “secondary reaction”) in series in a single reaction mixture. References to “first” and “second” invasive cleavage assays simply provides identifiers for distinguishing one invasive cleavage assay from another, without necessarily indicating one precedes the other.

A “reaction mixture” is a combination of reagents (e.g., oligonucleotides, target nucleic acids, enzymes, etc.) in a single reaction vessel.

As used herein, a “multiplex” assay is a type of assay that detects or measures multiple analytes (e.g., two or more nucleic acid sequences) in a single run of the assay. It is distinguished from procedures that measure one analyte per reaction mixture. A multiplex invasive cleavage assay is carried out by combining into a single reaction vessel the reagents for two or more different invasive cleavage assays. In some embodiments, the same species of fluorescent reporter is detected in each of the assays of the multiplex.

As used herein, the term “invasive cleavage structure” (or simply “cleavage structure”) refers to a structure comprising: (1) a target nucleic acid, (2) an upstream nucleic acid (e.g., an invasive probe oligonucleotide), and (3) a downstream nucleic acid (e.g., a primary probe oligonucleotide), where the upstream and downstream nucleic acids anneal to contiguous regions of the target nucleic acid, and where an overlap forms between the a 3′ portion of the upstream nucleic acid and duplex formed between the downstream nucleic acid and the target nucleic acid. An overlap occurs where one or more bases from the upstream and downstream nucleic acids occupy the same position with respect to a target nucleic acid base, whether the overlapping base(s) of the upstream nucleic acid are complementary with the target nucleic acid, and whether those bases are natural bases or non-natural bases. In some embodiments, the 3′ portion of the upstream nucleic acid that overlaps with the downstream duplex is a non-base chemical moiety such as an aromatic ring structure, as disclosed, for example, in U.S. Pat. No. 6,090,543. In some embodiments, one or more of the nucleic acids may be attached to each other, for example through a covalent linkage such as nucleic acid stem-loop, or through a non-nucleic acid chemical linkage (e.g., a multi-carbon chain).

As used herein, the term “flap endonuclease” or “FEN” (e.g., “FEN enzyme”) refers to a class of nucleolytic enzymes that act as structure-specific endonucleases on DNA structures with a duplex containing a single stranded 5′ overhang, or flap, on one of the strands that is displaced by another strand of nucleic acid, such that there are overlapping nucleotides at the junction between the single and double-stranded DNA. FEN enzymes catalyze hydrolytic cleavage of the phosphodiester bond 3′ adjacent to the junction of single and double stranded DNA, releasing the overhang, or “flap” (see Trends Biochem. Sci. 23:331-336 (1998) and Annu. Rev. Biochem. 73: 589-615 (2004)). FEN enzymes may be individual enzymes, multi-subunit enzymes, or may exist as an activity of another enzyme or protein complex, such as a DNA polymerase. A flap endonuclease may be thermostable. Examples of FEN enzymes useful in the methods disclosed herein are described in U.S. Pat. Nos. 5,614,402; 5,795,763; 6,090,606; and in published PCT applications identified by WO 98/23774; WO 02/070755; WO 01/90337; and WO 03/073067, each of which is incorporated by reference in its entirety. Examples of commercially available FEN enzymes include the Cleavase® enzymes (Hologic, Inc.).

As used herein, the term “probe” refers to an oligonucleotide that interacts with a target nucleic acid to form a detectable complex. Examples include invasive probes and primary probes. An “invasive probe” (sometimes “Invader Oligo”) refers to an oligonucleotide that hybridizes to a target nucleic acid at a location near the region of hybridization between a primary probe and the target nucleic acid, wherein the invasive probe oligonucleotide comprises a portion (e.g., a chemical moiety, or nucleotide, whether complementary to that target or not) that overlaps with the region of hybridization between the primary probe oligonucleotide and the target nucleic acid. The “primary probe” includes a target-specific region that hybridizes to the target nucleic acid, and further includes a “5′-flap” region that is not complementary to the target nucleic acid.

As used herein, the term “primary reaction” refers to enzymatic cleavage of a primary probe, whereby a cleaved 5′-flap is generated. The sequence of the cleaved 5′-flap will be the 5′-flap sequence of the primary probe (i.e., the sequence not complementary to the target nucleic acid), and one base at its 3′ terminus from the target-specific region (i.e., the sequence complementary to the target nucleic acid) of the primary probe.

As used herein, the term “secondary reaction” refers to enzymatic cleavage of a FRET cassette (following hybridization of a cleaved 5′-flap) to generate a detectable signal.

As used herein, the term “donor” refers to a moiety (e.g., a fluorophore) that absorbs at a first wavelength and emits at a second, longer wavelength. The term “acceptor” refers to a moiety such as a fluorophore, chromophore, or quencher and that can absorb some or most of the emitted energy from the donor when it is near the donor group (e.g., between 1-100 nm). An acceptor may have an absorption spectrum that overlaps the donor's emission spectrum. Generally, if the acceptor is a fluorophore, it then re-emits at a third, still longer wavelength; if it is a chromophore or quencher, it releases the energy absorbed from the donor without emitting a photon. In some preferred embodiments, alteration in energy levels of donor and/or acceptor moieties are detected (e.g., via measuring energy transfer, for example by detecting light emission) between or from donors and/or acceptor moieties). In some preferred embodiments, the emission spectrum of an acceptor moiety is distinct from the emission spectrum of a donor moiety such that emissions (e.g., of light and/or energy) from the moieties can be distinguished (e.g., spectrally resolved) from each other.

As used herein, “attached” (e.g., two things are “attached”) means chemically bonded together. For example, a fluorophore moiety is “attached” to a FRET cassette when it is chemically bonded to the structure of the FRET cassette.

As used herein, the term “FRET cassette” refers to an oligonucleotide, preferably a hairpin structure, that includes a donor moiety and a nearby acceptor moiety, where attachment of the donor and acceptor moieties to the same FRET cassette substantially suppresses (e.g., quenches) a detectable energy emission (e.g., a fluorescent emission). Cleavage of the FRET cassette by a FEN enzyme in a secondary reaction separates the donor and acceptor moieties with the result of relieving the suppression and permitting generation of a signal. In some embodiments, the donor and acceptor moieties interact by fluorescence resonance energy transfer (e.g., “FRET”). In other embodiments, the donor and acceptor of the FRET cassette interact by a non-FRET mechanism.

As used herein, an “interactive” label pair refers to a donor moiety and an acceptor moiety being attached to the same FRET cassette, and being in energy transfer relationship (i.e., whether by a FRET or a non-FRET mechanism) with each other. A signal (e.g., a fluorescent signal) can be generated when the donor and acceptor moieties are separated, for example by cleavage of the FRET cassette in a secondary reaction. Different FRET cassettes that specifically hybridize to different cleaved 5′-flaps can each include the same interactive label pair.

As used herein, emission from a donor moiety (e.g., a fluorophore) is “quenched” when the emission of a photon from the donor is prevented because an acceptor moiety (e.g., a quencher) is sufficiently close. For example, emission from a donor moiety is quenched when the donor moiety and the acceptor moiety are both attached to the same FRET cassette.

As used herein, the term “hybridize” or “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids.

As used herein, the term “Tm” is used in reference to the “melting temperature.” The melting temperature is the temperature at which a population of double-stranded nucleic acid molecules becomes half dissociated into single strands. The equation for calculating the Tm of nucleic acids is well known in the art.

As used herein, “specific” means pertaining to only one (or to only a particularly indicated group), such as having a particular effect on only one (or on only a particularly indicated group), or affecting only one (or only a particularly indicated group) in a particular way. For example, a cleaved 5′-flap specific for a FRET cassette will be able to hybridize to that FRET cassette and promote a cleavage reaction, but will not be able to hybridize to a different FRET cassette.

As used herein, the term “specifically hybridizes” means that under given hybridization conditions a probe or primer detectably hybridizes substantially only to the target sequence in a sample comprising the target sequence (i.e., there is little or no detectable hybridization to non-target sequences).

The term “thermostable” when used in reference to an enzyme, such as a FEN enzyme, indicates that the enzyme is functional or active (i.e., can perform catalysis) at an elevated temperature (i.e., at about 55° C. or higher). In some embodiments, the enzyme is functional or active at an elevated temperature of 65° C. or higher (e.g., 75° C., 85° C., 95° C., etc.).

As used herein, the terms “target nucleic acid” and “target sequence” refer to a nucleic acid that is to be detected or analyzed. Thus, the “target” is sought to be distinguished from other nucleic acids or nucleic acid sequences. For example, when used in reference to an amplification reaction, these terms may refer to the nucleic acid or portion of nucleic acid that will be amplified by the reaction, while when used in reference to a polymorphism (e.g., a mutation or nucleic acid sequence such as a genetic marker of drug resistance), they may refer to the portion of a nucleic acid containing a suspected polymorphism. When used in reference to an invasive cleavage reaction, these terms refer to a nucleic acid molecule containing a sequence that has at least partial complementarity with at least a first nucleic acid molecule (e.g. primary probe oligonucleotide) and also have at least partial complementarity with a second nucleic acid molecule (e.g. invasive probe oligonucleotide).

As used herein, the term “amplified” refers to an increase in the abundance of a molecule, moiety or effect. A target nucleic acid may be amplified, for example by in vitro replication, such as by PCR.

As used herein, the term “amplification method” when used in reference to nucleic acid amplification means a process of specifically amplifying the abundance of a nucleic acid of interest. Some amplification methods (e.g., polymerase chain reaction, or PCR) comprise iterative cycles of thermal denaturation, oligonucleotide primer annealing to template molecules, and nucleic acid polymerase extension of the annealed primers. Conditions and times necessary for each of these steps are well known in the art. Some amplification methods are conducted at a single temperature and are deemed “isothermal.” Accumulation of the products of amplification may be exponential or linear. Some amplification methods (“target amplification” methods) amplify the abundance of a target sequence by copying it many times (e.g., PCR, NASBA, TMA, strand displacement amplification, ligase chain reaction, LAMP, ICAN, RPA, SPA, HAD, etc.). Some amplification methods amplify the abundance of a nucleic acid species that may or may not contain the target sequence, the amplification of which indicates the presence of a particular target sequence in the reaction (e.g., rolling circle amplification, RAM amplification).

As used herein, the terms “polymerase chain reaction” and “PCR” refer to an enzymatic reaction in which a segment of DNA is replicated from a target nucleic acid in vitro. The reaction generally involves extension of a primer on each strand of a target nucleic acid with a template dependent DNA polymerase to produce a complementary copy of a portion of that strand. The chain reaction comprises iterative cycles of denaturation of the DNA strands, for example by heating, followed by cooling to allow primer annealing and extension, resulting in an exponential accumulation of copies of the region of the target nucleic acid that is flanked by and that includes the primer binding sites. When an RNA target nucleic acid is amplified by PCR, it is generally converted to a DNA copy strand with an enzyme capable of reverse transcription. Exemplary enzymes include MMLV reverse transcriptase, AMV reverse transcriptase, as well as other enzymes that will be familiar to those having an ordinary level of skill in the art.

The term “oligonucleotide” as used herein is defined as a molecule comprising two or more nucleotides (e.g., deoxyribonucleotides or ribonucleotides), preferably at least 5 nucleotides, more preferably at least about 10-15 nucleotides and more preferably at least about 15 to 30 nucleotides, or longer (e.g., oligonucleotides are typically less than 200 residues long (e.g., between 15 and 100 nucleotides), however, as used herein, the term is also intended to encompass longer polynucleotide chains). The exact size will depend on many factors, which in turn depend on the ultimate function or use of the oligonucleotide. Oligonucleotides are often referred to by their length. For example, a 24 residue oligonucleotide is referred to as a “24-mer.” Oligonucleotides can form secondary and tertiary structures by self-hybridizing or by hybridizing to other polynucleotides. Such structures can include, but are not limited to, duplexes, hairpins, cruciforms, bends, and triplexes. Oligonucleotides may be generated in any manner, including chemical synthesis, DNA replication, reverse transcription, PCR, or a combination thereof. In some embodiments, oligonucleotides that form invasive cleavage structures are generated in a reaction (e.g., by extension of a primer in an enzymatic extension reaction).

As used herein, a “signal” is a detectable quantity or impulse of energy, such as electromagnetic energy (e.g., light). Emission of light from an appropriately stimulated fluorophore is an example of a fluorescent signal. In some embodiments, “signal” refers to the aggregated energy detected in a single channel of a detection instrument (e.g., a fluorometer).

As used herein, a “background” signal is the signal (e.g., a fluorescent signal) generated under conditions that do not permit a target nucleic acid-specific reaction to take place. For example, signal generated in a secondary reaction that includes a FRET cassette and FEN enzyme, but not a cleaved 5′-flap would produce a background signal. In some instances, a background signal is measured in a “negative control” trail that omits the target nucleic acid.

As used herein a “channel” of an energy sensor device, such as a device equipped with an optical energy sensor, refers to a pre-defined band of wavelengths that can be detected or quantified to the exclusion of other bands of wavelengths. For example, one detection channel of a fluorometer might be capable of detecting light energy emitted by one or more fluorescent labels over a range of wavelengths as a single event. Light emitted as the result of fluorescence can be quantified as relative fluorescence units (RFU) at a given wavelength, or over a band of wavelengths.

As used herein, the term “allele” refers to a variant form of a given sequence (e.g., including but not limited to, genes containing one or more single nucleotide polymorphisms or “SNPs”). A large number of genes are present in multiple allelic forms in a population. A diploid organism carrying two different alleles of a gene is said to be heterozygous for that gene, whereas a homozygote carries two copies of the same allele.

The term “wild-type” (also “WT” herein) refers to a gene or gene product that has the characteristics of that gene or gene product when isolated from a naturally occurring source. A wild-type gene is that which is most frequently observed in a population and is thus arbitrarily designated the “normal” or “wild-type” form of the gene. In contrast, the terms “modified,” “mutant” (also “Mut” herein), and “variant” refer to a gene or gene product that displays modifications in sequence and or functional properties (i.e., altered characteristics) when compared to the wild-type gene or gene product.

As used herein, a “threshold” or “threshold cutoff” refers to a quantitative limit used for interpreting experimental results, where results above and below the cutoff lead to opposite conclusions. For example, a measured signal falling below a cutoff may indicate the absence of a particular target, but a measured signal that exceeds the same cutoff may indicate the presence of that target. By convention, a result that meets a cutoff (i.e., has exactly the cutoff value) is given the same interpretation as a result that exceeds the cutoff.

As used herein, a “threshold cycle number” refers to indicia of amplification that measure the time or cycle number when a real-time run curve signal crosses an arbitrary value or threshold. “TTime” and “Ct” determinations are examples of threshold-based indicia of amplification. Other methods involve performing a derivative analysis of the real-time run curve. For this disclosure, TArc and OTArc also can be used to determine when a real-time run curve signal crosses an arbitrary value (e.g., corresponding to a maximum or minimum angle in curvature, respectively). Methods of Time determination are disclosed in U.S. Pat. No. 8,615,368; methods of Ct determination are disclosed in EP 0640828 B1; derivative-based methods are disclosed in U.S. Pat. No. 6,303,305; and methods of TArc and OTArc determination are disclosed in U.S. Pat. No. 7,739,054. Those having an ordinary level of skill in the art will be aware of variations that also can be used for determining threshold cycle numbers.

As used herein, a “reaction vessel” or “reaction receptacle” is a container for containing a reaction mixture. Examples include individual wells of a multiwell plate, and plastic tubes (e.g., including individual tubes within a formed linear array of a multi-tube unit, etc.). However, it is to be understood that any suitable container may be used for containing the reaction mixture.

As used herein, “permitting” a reaction to take place means that reagents and conditions are provided by reaction mixture to test for the presence of a particular nucleic acid (e.g., a target DNA, or a cleaved 5′-flap), which may or may not be present in the reaction mixture. For example, “permitting” a primary reaction of an invasive cleavage assay to take place means that a reaction mixture includes an invasive probe, a primary probe that includes a 5′-flap sequence, and a FEN enzyme under appropriate buffer and temperature conditions to allow cleavage of the primary probe and release of a cleaved 5′-flap if a target DNA also is available in the reaction mixture to participate in the primary reaction. Similarly, “permitting” a secondary reaction of an invasive cleavage assay to take place means that a reaction mixture includes a FRET cassette and a FEN enzyme under appropriate buffer and temperature conditions to allow cleavage of the FRET cassette if a cleaved 5′-flap specific for the FRET cassette also is available in the reaction mixture to participate in the secondary reaction. Still further, temperature conditions “permitting” (or that “permit” or are “permissive” for) a reaction to take place are temperature conditions that are conducive for conducting or allowing the reaction to proceed.

### DETAILED DESCRIPTION

### Introduction

Disclosed herein is a generalized method for determining the presence or absence of a target nucleic acid sequence, where the method can involve comparing Ct values determined for two different target nucleic acid sequences in the same amplification product (e.g., even using opposite strands for detection of the different targets). Markers for the different targets can include nucleotide bases that vary in composition at one or more positions in the target sequence. One marker can be a single-nucleotide polymorphism (SNP) that may be present. The other marker can be an invariant sequence, such as a wild-type sequence that serves as a positive control for amplification and detection procedures. In these embodiments, the wild-type marker and the SNP are not at the same position in the amplification product. It can be determined that the SNP is present if Ct values for the wild-type and SNP markers are substantially the same, or within a narrow range of each other. The narrow range typically will be 0-4 cycles. When the difference between Ct values (ΔCt) exceeds this range, and if the invariant sequence serving as the positive control is detected, then the sample can be judged as substantially not including nucleic acid containing the SNP. In some embodiments, the two different target nucleic acid sequences are detected using invasive cleavage reactions.

Also disclosed herein are oligonucleotides, compositions, kits, and methods that can be used to amplify and detect genetic markers of macrolide resistance in M. genitialium. In certain preferred embodiments, a single amplicon synthesized in an in vitro nucleic acid amplification reaction is used for detecting both a marker of macrolide resistance, and a wild-type M. genitialium sequence that serves as a positive control in the amplification and detection assay. Optionally, the genetic marker for macrolide resistance and the wild-type sequence can be detected on complementary strands of the same double-stranded amplicon (e.g., a PCR product).

The disclosed method can be used for detecting and identifying M. genitialium by testing naïve samples, but preferably is used as a reflex assay that particularly reports the presence or absence of macrolide resistance in a sample already known to contain M. genitialium. The reflex assay approach yielded a superior positive predictive value for the assay. Positive predictive value correlates with prevalence. By testing a reflex sample set, we are only using the disclosed assay for testing samples positive for M. genitalium, thereby maximizing the positive predictive value of the assay. Indeed, superior results were achieved when samples undergoing testing already were known to contain M. genitialium, even though a wild-type M. genitialium nucleic acid sequence was amplified and detected as a positive control in the procedure. While not wishing to be bound by any particular theory of operation, the improved result is believed due to the prevalence of macrolide-resistant organisms in the clinical population being tested. There is, however, flexibility in the assay protocol. More particularly, detection of the wild-type M. genitialium nucleic acid sequence as a positive amplification and detection control (i.e., in the same amplicon used for detecting drug resistance markers) also can be used for indicating the presence of M. genitialium in the absence of the drug resistance marker.

Procedures for identifying macrolide-resistant M. genitialium can be carried out in different ways. For example, there can be separate assays that independently identify the presence of nucleic acids characteristic of M. genitialium and the macrolide resistance marker (e.g., no shared oligonucleotides). Alternatively, standard microbiological culture techniques can be used to indicate the presence of M. genitialium in a sample that subsequently is tested for the presence of nucleic acid marker(s) of macrolide resistance. Alternatively, a single assay can be used for detecting and identifying nucleic acid markers indicative of M. genitialium and macrolide resistance.

### Description of Certain Embodiments

Disclosed is a technique that synthesizes multiple copies of an M. genitialium target nucleic acid and detects the sequences of wild-type and/or macrolide-resistant variants. This can involve a pair of oligonucleotides, where one oligonucleotide is configured to hybridize to a sense strand of an M. genitalium nucleic acid and the other is configured to hybridize to an anti-sense strand of an M. genitalium nucleic acid. Such oligonucleotides include primer pairs for PCR or other forms of amplification. Alternatively, such oligonucleotides can be primary probes or invasive probes that hybridize to opposite strands of the same double-stranded PCR product produced using the M. genitalium 23S ribosomal nucleic acid as the template. Here the PCR product includes both wild-type sequence and sequence associated with resistance to macrolide antibiotics. The primary probes that detect these sequences (i.e., markers indicating wild-type and macrolide resistance sequences) can hybridize to different DNA strands of the amplified nucleic acid.

The disclosed method or assay can be used as a reflex test to a positive result from a different assay that detects M. genitalium to determine if an infection with this organism is sensitive or resistant to azithromycin. Stated differently, the disclosed method can be used for testing samples already known to contain M. genitialium bacteria. Patients identified as having azithromycin-resistant infections can be diverted to treatment with fluoroquinolones, the last known antibiotic class that is effective against M. genitialium.

Optionally, M. genitialium-specific amplification products are detected at the end of an amplification reaction using an “end-point” formatted assay.

Optionally, synthesis of M. genitialium-specific amplification products can be monitored periodically as the amplification reaction is taking place. This is sometimes referred to as a “real-time” formatted assay. Preferably, the assay uses the combination of real-time reverse transcription PCR and an invasive cleavage assay to detect mutations in the 23S rRNA of M. genitalium that confer resistance to the macrolide antibiotic azithromycin. The combination of PCR amplification with real-time invasive cleavage detection is sometimes referred to as the “Invader Plus®” technique.

In some embodiments, one or more oligonucleotides, such as a primer set (defined as at least two primers configured to generate or detect an amplicon from a target sequence) or a primer set and an additional oligonucleotide (e.g., a detection oligonucleotide) which is optionally non-extendible and/or labeled (e.g., for use as a primary probe or part of a probe system that includes a FRET cassette), are configured to hybridize to an amplification product of M. genitalium 23S ribosomal nucleic acid. In some embodiments, the primer set includes at least one reverse primer configured to hybridize to the 23S rRNA of M. genitalium, and at least one forward primer configured to hybridize to an extension product of the reverse primer using the ribosomal nucleic acid of M. genitalium as the template. When present, the additional oligonucleotide (e.g., a detection oligonucleotide such as a primary probe, or an invasive probe) can be configured to hybridize to an amplicon produced by the primer set. In some embodiments, one of the primers fuctions as an invasive probe for one of the primary probes.

In some embodiments, a plurality of oligonucleotides, optionally non-extendible and/or labeled (e.g., for use as primary probes, FRET cassettes, etc.), are provided which collectively hybridize to one or more sequences within an M. genitalium nucleic acid amplification product. In some embodiments, a sequence characteristic of wild-type M. genitalium is detected in the same amplification product that also is used for detecting macrolide resistance markers. In some embodiments, a plurality of oligonucleotides, such as a plurality of primer sets or a plurality of primer sets and additional oligonucleotides (e.g., detection oligonucleotides) which are optionally non-extendible and/or labeled (e.g., for use as a primary probe, optionally as part of a probe system, such as together with a FRET cassette), are provided which collectively hybridize to opposite strands of a double-stranded amplification product. In some embodiments, amplification or detection of the sequence indicative of M. genitalium discriminates the presence of M. genitalium from many other Mycoplasma species. Optionally, amplification or detection of the sequence indicative of M. genitalium can be highly specific for M. genitalium, so that nucleic acids from no other known organisms are detected.

In some embodiments, one or more oligonucleotides in a set, kit, composition, or reaction mixture include one or more methylated cytosine (e.g., 5-methylcytosine) residues. In some embodiments, at least about half of the cytosines in an oligonucleotide are methylated. In some embodiments, all or substantially all (e.g., all but one or two) of the cytosines in an oligonucleotide are methylated. For example, one or more cytosines at the 3′-end or within 2, 3, 4, or 5 bases of the 3′-end are unmethylated.

M. genitalium macrolide resistance can be assessed using reverse-transcription PCR of M. genitalium 23S rRNA, with invasive cleavage detection to permit real-time monitoring of amplicon synthesis. To detect mutations at either of base locations 2058 or 2059 (E. coli numbering in region V of the 23S rRNA), which have been shown to be associated with M. genitalium macrolide resistance (see Couldwell et al., Infect. Drug Resist. 8:147-161 (2015)), a single invasive probe was used in combination with four primary probes, each having the same attached 5′-flap sequence. Macrolide resistance is indicated when there is an A to G transition at position 2059. Alternatively, macrolide resistance is indicated when the naturally occurring A residue at position 2058 is replaced by any of G, C, or T. Either of these conditions (i.e., mutation at one of two adjacent nucleotide positions) can result in macrolide resistance, and it is unnecessary for both positions to be mutated simultaneously to produce the drug-resistant condition. Released 5′-flaps resulting from cleavage of any of the different primary probes specific for one of the macrolide resistance markers can interact with a shared (i.e., the same) FRET cassette to promote a secondary cleavage reaction resulting in release of a fluorophore (i.e., removal from attachment to a FRET cassette that also harbors a quencher). In this way any genotype being associated with macrolide resistance can be indicated by a single type of fluorescent signal (e.g., a FAM signal).

In some embodiments, an oligonucleotide is provided that includes a label and/or is non-extendable. Such an oligonucleotide can be used as a probe or as part of a probe system (e.g., as a FRET cassette in combination with a target-binding detection oligonucleotide). In some embodiments, the FRET cassette has a sequence corresponding to one of the FRET cassettes disclosed herein. In some embodiments, the label is a non-nucleotide label. Example labels include compounds that emit a detectable light signal, such as fluorophores or luminescent (e.g., chemiluminescent) compounds that can be detected in a homogeneous mixture. More than one label, and more than one type of label, can be present on a particular probe, or detection can rely on using a mixture of probes in which each probe is labeled with a compound that produces a detectable signal (see e.g., U.S. Pat. Nos. 6,180,340 and 6,350,579). Labels can be attached to a probe by various means including covalent linkages, chelation, and ionic interactions. In some embodiments the label is covalently attached. For example, in some embodiments, a detection probe has an attached chemiluminescent label such as, for example, an acridinium ester (AE) compound (see e.g., U.S. Pat. Nos. 5,185,439; 5,639,604; 5,585,481; and 5,656,744). A label, such as a fluorescent or chemiluminescent label, can be attached to the probe by a non-nucleotide linker (see e.g., U.S. Pat. Nos. 5,585,481; 5,656,744; and 5,639,604). In some embodiments, an oligonucleotide is provided that is non-extendible and hybridizes to a site in an M. genitalium nucleic acid that overlaps the hybridization site of an additional oligonucleotide in a kit or composition, such as an amplification primer. Hybridization of such oligonucleotides can form a substrate for a structure-specific nuclease, for example, as part of the detection mechanism in endpoint or real-time nucleic acid assays employing invasive cleavage detection assays.

In some embodiments, a labeled oligonucleotide (e.g., including a fluorescent label) further includes a second label that interacts with the first label. For example, the second label can be a quencher. Such probes can be used (e.g., in TaqMan™ assays) where hybridization of the probe to a target or amplicon followed by nucleolysis by a polymerase including 5′-3′ exonuclease activity results in liberation of the fluorescent label and thereby increased fluorescence, or fluorescence independent of the interaction with the second label. Such probes can also be used to label FRET cassettes, which can be components of Invader® or Invader Plus® nucleic acid assays.

Examples of interacting donor/acceptor label pairs that can be used in connection with the disclosure include fluorescein/tetramethylrhodamine, IAEDANS/fluororescein, EDANS/DABCYL, coumarin/DABCYL, fluorescein/fluorescein, BODIPY® FL/BODIPY® FL, fluorescein/DABCYL, lucifer yellow/DABCYL, BODIPY®/DABCYL, eosine/DABCYL, erythrosine/DABCYL, tetramethylrhodamine/DABCYL, Texas Red/DABCYL, CY5/BHQ1®, CY5/BHQ2®, CY3/BHQ1®, CY3/BHQ2® and fluorescein/QSY7® dye. Those having an ordinary level of skill in the art will understand that when donor and acceptor dyes are different, energy transfer can be detected by the appearance of sensitized fluorescence of the acceptor or by quenching of donor fluorescence. Non-fluorescent acceptors such as DABCYL and the QSY7® dyes advantageously eliminate the potential problem of background fluorescence resulting from direct (i.e., non-sensitized) acceptor excitation. Exemplary fluorophore moieties that can be used as one member of a donor-acceptor pair include fluorescein, HEX, ROX, and the CY dyes (such as CY5). Exemplary quencher moieties that can be used as another member of a donor-acceptor pair include DABCYL BLACKBERRY QUENCHER® which are available from Berry and Associates (Dexter, Mich.), and the BLACK HOLE QUENCHER® moieties which are available from Biosearch Technologies, Inc., (Novato, Calif.). One of ordinary skill in the art will be able to use appropriate pairings of donor and acceptor labels for use in various detection formats (e.g., FRET, TaqMan™, Invader®, etc.).

As discussed above, a detection oligonucleotide (e.g., invasive probe, primary probe, or labeled FRET cassette) is non-extendable. For example, the oligonucleotide can be rendered non-extendable by a 3′-adduct (e.g., 3′-phosphorylation or 3′-alkanediol), having a 3′-terminal 3′-deoxynucleotide (e.g., a terminal 2′,3′-dideoxynucleotide), having a 3′-terminal inverted nucleotide (e.g., in which the last nucleotide is inverted such that it is joined to the penultimate nucleotide by a 3′ to 3′ phosphodiester linkage or analog thereof, such as a phosphorothioate), or having an attached fluorophore, quencher, or other label that interferes with extension (possibly but not necessarily attached via the 3′ position of the terminal nucleotide). In some embodiments, the 3′-terminal nucleotide is not methylated. In some embodiments, a detection oligonucleotide includes a 3′-terminal adduct such as a 3′-alkanediol (e.g., hexanediol).

In some embodiments, an oligonucleotide such as a detection oligonucleotide is configured to specifically hybridize to an M. genitalium amplicon. The oligonucleotide can include or consist of a target-hybridizing sequence sufficiently complementary to the amplicon for specific hybridization. The target-hybridizing sequence can be joined at its 5′-end to a nucleotide sequence that is not complementary to the amplicon being detected.

Also provided are kits for performing the methods described herein. A kit in accordance with the present disclosure includes at least one or more of the following: an amplification oligonucleotide combination capable of amplifying an M. genitalium 23S ribosomal nucleic acid; and at least one detection probe oligonucleotide as described herein for determining the presence or absence of one or more macrolide resistance markers in the M. genitalium amplification product. In some embodiments, any oligonucleotide combination described herein is present in the kit. As well, any of the disclosed oligonucleotides can be combined in any combination and packaged together in a kit. The kits can further include a number of optional components such as, for example, capture probes (e.g., poly-(k) capture probes as described in US 2013/0209992), as well as a primary probe that detects a wild-type M. genitalium sequence in the same amplicon harboring the macrolide resistance marker.

Other reagents that can be present in the kits include reagents suitable for performing in vitro amplification such as, for example, buffers, salt solutions, appropriate nucleotide triphosphates (e.g., dATP, dCTP, dGTP, and one or both of dTTP or dUTP; and/or ATP, CTP, GTP and UTP), and/or enzymes (e.g., a thermostable DNA polymerase, and/or reverse transcriptase and/or RNA polymerase and/or FEN enzyme), and will typically include test sample components, in which an M. genitalium target nucleic acid may or may not be present. In addition, for a kit that includes a detection probe together with an amplification oligonucleotide combination, selection of amplification oligonucleotides and detection probe oligonucleotides for a reaction mixture are linked by a common target region (i.e., the reaction mixture will include a probe that hybridizes to a sequence amplifiable by an amplification oligonucleotide combination of the reaction mixture). In certain embodiments, the kit further includes a set of instructions for practicing methods in accordance with the present disclosure, where the instructions can be associated with a package insert and/or the packaging of the kit or the components thereof.

Any method disclosed herein is also to be understood as a disclosure of corresponding uses of materials involved in the method directed to the purpose of the method. Any of the oligonucleotides including an M. genitalium sequence and any combinations (e.g., kits and compositions, including but not limited to reaction mixtures) including such an oligonucleotide are to be understood as also disclosed for use in detecting or quantifying macrolide-resistant M. genitalium, and for use in the preparation of a composition for detecting macrolide-resistant M. genitalium.

Broadly speaking, methods can employ one or more of the following elements: target capture, in which M. genitalium nucleic acid (e.g., from a sample, such as a clinical sample) is annealed to a capture oligonucleotide (e.g., a specific or nonspecific capture oligonucleotide); isolation (e.g., washing, to remove material not associated with a capture oligonucleotide); amplification; and amplicon detection, which for example can be performed in real-time with amplification. Certain embodiments involve each of the foregoing steps. Certain embodiments involve exponential amplification, optionally with a preceding linear amplification step. Certain embodiments involve exponential amplification and amplicon detection. Certain embodiments involve any two of the components listed above. Certain embodiments involve any two elements listed adjacently above (e.g., washing and amplification, or amplification and detection).

In some embodiments, amplification includes (1) contacting a nucleic acid sample with at least two oligonucleotides for amplifying a segment of M. genitalium 23S ribosomal nucleic acid, where the amplified segment includes positions corresponding to positions 2058 and 2059 of region V in E. coli 23S rRNA. The oligonucleotides can include at least two amplification oligonucleotides (e.g., one oriented in the sense direction and one oriented in the antisense direction for exponential amplification); (2) performing an in vitro nucleic acid amplification reaction, where any M. genitalium target nucleic acid present in the sample is used as a template for generating an amplification product; and (3) detecting the presence or absence of markers of macrolide resistance in the amplification product, thereby determining the presence or absence of macrolide-resistant M. genitalium in the sample. The markers of macrolide resistance include a transition from A to G at position 2059, and a change from A to any of G, C, or T at position 2058.

A detection method in accordance with the present disclosure can further include the step of obtaining the sample to be subjected to subsequent steps of the method. In certain embodiments, “obtaining” a sample to be used includes, for example, receiving the sample at a testing facility or other location where one or more steps of the method are performed, and/or retrieving the sample from a location (e.g., from storage or other depository) within a facility where one or more steps of the method are performed.

Exponentially amplifying a target sequence can utilize an in vitro amplification reaction using at least two amplification oligonucleotides that flank a target region to be amplified. In some embodiments, at least two amplification oligonucleotides as described above are provided. The amplification reaction can be temperature-cycled or isothermal. Suitable amplification methods include, for example, replicase-mediated amplification, polymerase chain reaction (PCR), ligase chain reaction (LCR), strand-displacement amplification (SDA), and transcription-mediated amplification (TMA).

A detection step can be performed using any of a variety of known techniques to detect a signal specifically associated with the amplified target sequence, such as by hybridizing the amplification product with a labeled detection probe and detecting a signal resulting from the labeled probe (including from label released from the probe following hybridization in some embodiments). In some embodiments, the labeled probe includes a second moiety, such as a quencher or other moiety that interacts with the first label, as discussed above. The detection step can also provide additional information on the amplified sequence, such as all or a portion of its nucleic acid sequence. Detection can be performed after the amplification reaction is completed, but preferably is performed simultaneously with amplifying the target region (e.g., in real-time). In one embodiment, the detection step allows homogeneous detection (e.g., detection of the hybridized probe without removal of unhybridized probe from the mixture (see e.g., U.S. Pat. Nos. 5,639,604 and 5,283,174)). In some embodiments, the nucleic acids are associated with a surface that results in a physical change, such as a detectable electrical change. Amplified nucleic acids can be detected by concentrating them in or on a matrix and detecting the nucleic acids or dyes associated with them (e.g., an intercalating agenit such as ethidium bromide or cyber green), or detecting an increase in dye associated with nucleic acid in solution phase. Other methods of detection can use nucleic acid detection probes configured to hybridize to a sequence in the amplified product and detecting the presence of the probe:product complex, or by using a complex of probes that can amplify the detectable signal associated with the amplified products (e.g., U.S. Pat. Nos. 5,424,413; 5,451,503; and 5,849,481; each incorporated by reference herein). Directly or indirectly labeled probes that specifically associate with the amplified product provide a detectable signal that indicates the presence of the target nucleic acid in the sample. In particular, the amplified product will contain a target sequence in or complementary to a sequence in the M. genitalium chromosome, and a probe will bind directly or indirectly to a sequence contained in the amplified product to indicate the presence of macrolide-resistant M. genitalium nucleic acid in the tested sample.

In embodiments that detect the amplified product near or at the end of the amplification step, a linear detection probe can be used to provide a signal to indicate hybridization of the probe to the amplified product. One example of such detection uses a luminescentally labeled probe that hybridizes to target nucleic acid. The luminescent label can then be hydrolyzed from non-hybridized probe. Detection is performed by chemiluminescence using a luminometer (see, e.g., International Patent Application Pub. No. WO 89/002476). In other embodiments that use real-time detection, the detection probe can be a hairpin probe such as a molecular beacon, molecular torch, or hybridization switch probe that is labeled with a reporter moiety that is detected when the probe binds to amplified product. Such probes can include target-hybridizing sequences and non-target-hybridizing sequences. Various forms of such probes are described, for example, in U.S. Pat. Nos. 5,118,801; 5,312,728; 5,925,517; 6,150,097; 6,849,412; 6,835,542; 6,534,274; and 6,361,945; and US Patent Application Pub. Nos. 2006/0068417A1 and 2006/0194240A1).

Invasive cleavage assays can be used for detecting specific target sequences in unamplified, as well as amplified DNA (e.g., PCR product(s)), including genomic DNA, cDNA prepared by reverse transcribing RNA, or an amplicon thereof. The primary probe and the invasive probe hybridize in tandem to the target nucleic acid to form an overlapping structure. An unpaired “flap” is included on the 5′-end of the primary probe. A cleavage agent (e.g., a FEN enzyme, such as the Cleavase® enzymes available from Hologic, Inc.) recognizes the overlap and cleaves off the unpaired 5′-flap. The fragment, sometimes referred to as a “liberated flap” or “cleaved 5′-flap” or simply a “flap” can then itself interact with a secondary probe such as a FRET cassette (e.g., by participating as an invasive probe in a subsequent reaction that generates a detectable signal (e.g., a fluorescent signal)). Such embodiments are described in U.S. Pat. Nos. 5,846,717, 5,985,557, 5,994,069, 6,001,567, and 6,090,543, WO 97/27214, WO 98/42873, Nat. Biotech., 17:292 (1999), PNAS, 97:8272 (2000), and WO 2016/179093. More specifically, this cleaved product serves as an invasive probe on a FRET cassette in a secondary reaction to again create a structure recognized by the structure-specific enzyme. When the two labels on a single FRET cassette are separated by cleavage, a detectable fluorescent signal above background fluorescence is produced. Consequently, cleavage of the second invasive cleavage structure results in an increase in fluorescence, thereby indicating the presence of the target sequence. More specifically, a plurality of invasive cleavage reactions combined in a single reaction mixture can be used for the multiplex applications disclosed herein.

The disclosed assay preferably uses a target capture step to isolate 23S rRNA from M. genitialium, then reverse transcription PCR with real-time invasive cleavage detection to amplify and detect DNA copies of the 23S rRNA. A mixture of invasive probes and a FRET cassette can be used to interrogate base positions 2058 and 2059, which are mutated in M. genitialium that are resistant to azithromycin. The assay can report both wild-type (azithromycin-sensitive) and mutated (azithromycin-resistant) sequences, either alone or in mixtures with exceedingly high accuracy. Conversely, other approaches (e.g., melt curve analysis, or nucleic acid sequencing) can have difficulty distinguishing wild-type from drug-resistant mutant sequences in mixed infections. It was discovered during development of the present technique that mixed infections of wild-type and drug-resistant mutant M. genitialium are common in patient populations. Importantly, the disclosed technique can be used for detecting the genetic markers of macrolide resistance, even among a background of wild-type M. genitialium sequences that would be present in a mixed infection.

Briefly, the target capture method used in the presently disclosed assay employed an oligonucleotide probe immobilized directly to a magnetically attractable solid support (i.e., the “immobilized probe”) and a “capture probe” (or sometimes “target capture probe” or “target capture oligonucleotide”) that bridged the immobilized probe and the 23S M. genitialium target ribosomal nucleic acid to form a hybridization complex that could be separated from other components in the mixture. An illustrative instrument work station that can be used to carry out such a purification step is disclosed by Acosta et al., in U.S. Pat. No. 6,254,826, the disclosure of which is incorporated by reference. The capture probe is preferably designed so that the melting temperature of the capture probe:target nucleic acid hybrid is greater than the melting temperature of the capture probe:immobilized probe hybrid. In this way, different sets of hybridization assay conditions can be employed to facilitate hybridization of the capture probe to the target nucleic acid prior to hybridization of the capture probe to the immobilized oligonucleotide, thereby maximizing the concentration of free probe and providing favorable liquid phase hybridization kinetics. This “two-step” target capture method is disclosed by Weisburg et al., U.S. Pat. No. 6,110,678. In some embodiments, the 23S M. genitalium target ribosomal nucleic acid is captured onto the solid support by direct interaction (e.g., hybridization) with the immobilized probe, and there is no requirement for a target capture probe. Other target capture schemes readily adaptable to the present technique are well known in the art and include, without limitation, those disclosed by the following: Dunn et al., Methods in Enzymology, “Mapping viral mRNAs by sandwich hybridization,” 65(1):468-478 (1980); Ranki et al., U.S. Pat. No. 4,486,539; Stabinsky, U.S. Pat. No. 4,751,177; and Becker et al., U.S. Pat. No. 6,130,038.

Isolation can follow capture, wherein the complex on the solid support is separated from other sample components. Isolation can be accomplished by any appropriate technique (e.g., washing a support associated with the M. genitalium-target-sequence one or more times (e.g., 2 or 3 times) to remove other sample components and/or unbound oligonucleotide). In embodiments using a particulate solid support, such as paramagnetic beads, particles associated with the M. genitalium-target can be suspended in a washing solution and retrieved from the washing solution, in some embodiments by using magnetic attraction. To limit the number of handling steps, the M. genitalium target nucleic acid can be amplified by simply mixing the M. genitalium target sequence in the complex on the support with amplification oligonucleotides and proceeding with amplification steps.

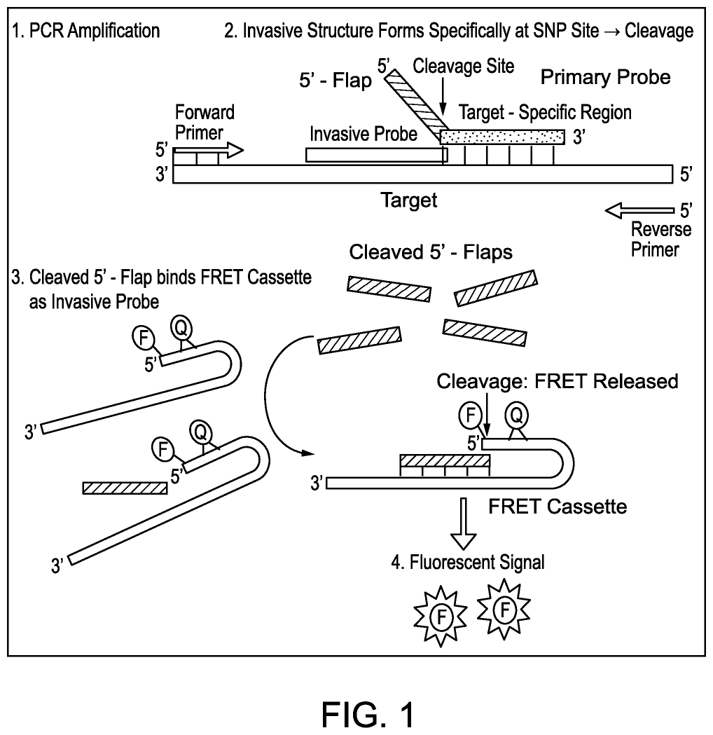

Essential features of some real-time amplification and detection schemes that can be employed in the disclosed assay are presented in FIG. 1. The target nucleic acid is amplified using a paired set of forward and reverse primers in a reaction mixture that further includes a primary probe specific for a wild-type sequence, an invasive probe and allele-specific primary probes that detect macrolide resistance markers, and a FRET cassette. The invasive probe and the allele-specific primary probes specific for macrolide resistance markers hybridize to one strand of an amplified nucleic acid during an annealing step of PCR to form a base pair overlap, such as a 1-2 base pair overlap, at a mutation site. Cleavase® enzyme (e.g., a flap endonuclease, or “FEN” enzyme commercially available from Hologic, Inc.) releases a cleaved 5′-flap oligonucleotide (sometimes “5′-flap oligo”) from an allele-specific primary probe only if there is perfect complementarity at the overlap site. Cleaved 5′-flap oligonucleotides can then bind to the FRET cassette as secondary invasive probes. Cleavase® enzyme activity separates fluorophore from quencher of the FRET cassette, thereby permitting the fluorophore to emit a detectable fluorescent signal. Fluorescence can be detected in real-time using real-time quantitative PCR instrumentation.

Preferred reactions that amplified and detected the M. genitialium macrolide resistance marker further included oligonucleotides that detected a wild-type M. genitialium sequence within the same amplification product that was used for detecting the macrolide resistance marker, if present. Detection of the wild-type sequence served as a positive control in the procedure to verify the presence of nucleic acids derived from M. genitialium (i.e., both macrolide-resistant and macrolide-sensitive strains). If negative results were obtained in the assay for detecting the drug resistance marker, detection of a signal indicating that the positive control sequence amplified served to validate the negative result by confirming the assay was operational. Those having an ordinary level of skill in the art will appreciate that affirmative detection of an amplification signal from a positive control nucleic acid indicates that the reaction was competent to amplify and detect target nucleic acids. Optionally, nucleic acid sequences indicating the presence of wild-type M. genitialium and macrolide-resistant M. genitialium are detected on opposite (i.e., complementary) strands of a nucleic acid amplification product.

### Interpretation of Results in the Multiplex Reactions

The disclosed technique can be used for detecting single nucleotide polymorphisms (SNPs) by comparing Ct values measured for the SNP marker (e.g., indicating macrolide resistance) and the Ct value measured for the positive control sequence (e.g., a wild-type sequence) present in the same amplicon, allowing for detection of the different sequences on complementary strands of the same amplification product. Thus, complementary strands of a DNA amplification product synthesized in a PCR reaction mixture can be used for detecting a SNP and a wild-type sequence, and Ct values determined for each of those targets can be compared to determine the presence or absence of the SNP in the amplicon.

In embodiments employing multiplex detection of both wild-type (i.e., positive control) and macrolide resistance marker sequences in the same amplicon (e.g., opposite strands of the same double-stranded amplification product), a first fluorophore (e.g., HEX, below) indicated detection of the positive control sequence, and a different second fluorophore (e.g., FAM, below) indicated detection of one of the SNPs for a macrolide resistance marker. Both detection systems had substantially similar amplification efficiencies when the probes bound to their targets. When the same amplicon includes both the wild-type (e.g., positive control) sequence and a macrolide resistance marker (e.g., any one of the SNPs), the Ct values are expected to be substantially similar in the real-time amplification reaction. Determining a difference between these Ct values can indicate the presence or absence of the SNP in the amplicon. For example, if the absolute value of the difference between Ct values (e.g., |Ct(Hex)−Ct(FAM)|, or simply “ΔCt”) is close to 0 cycles (e.g., any of 0 cycles, 1 cycle, 2 cycles, 3 cycles, or 4 cycles; or any of 0-4 cycles, 0-3 cycles, 0-2 cycles, or 0-1 cycles), then the sample can be judged as being positive for nucleic acids of macrolide-resistant M. genitalium. Conversely, if the SNP is not present, then emergence of the FAM signal is substantially delayed, but the HEX signal (i.e., the positive control) otherwise indicates the presence of the M. genitalium target nucleic acid sequence. In this instance, a sample can be judged as comprising macrolide-sensitive (sometimes “macrolide-susceptible”) M. genitalium if a ΔCt value substantially greater than 0 because the two run curves are substantially different (e.g., by at least about 5 cycles, at least about 6 cycles, at least about 8 cycles, or even at least about 10 cycles).

Simply stated, a method for determining the presence or absence of macrolide resistant M. genitalium can involve comparing Ct values determined for wild-type and drug resistance markers in the same amplification product (e.g., even using opposite strands for detection of the different targets). It can be determined that nucleic acids of macrolide-resistant M. genitalium are present if the wild-type sequence is detected and the Ct values (i.e., for wild-type and drug resistance markers) are substantially the same, or within a narrow range of each other. The narrow range typically will be 0-4 cycles. When the ΔCt value exceeds this range, and when the wild-type (i.e., the positive control) sequence is detected, the sample can be judged as containing substantially only macrolide-sensitive M. genitalium.

According to a simplified analysis presented in Table 1, whether the difference in Ct values is positive or negative can also indicate whether a sample includes nucleic acids of macrolide-sensitive or macrolide-resistant M. genitalium. Here the ΔCt value is conventionally calculated by subtracting the Ct value measured for the drug resistance marker from the Ct value measured for the wild-type sequence in the same amplification product (allowing for detection of the different targets on complementary strands). The calculated ΔCt value will always be negative when testing nucleic acids of macrolide-sensitive M. genitalium because the delayed emergence of the signal indicating detection of the drug resistance marker yields a higher Ct value. Conversely, the calculated ΔCt value will always be positive when testing nucleic acids of macrolide-resistant M. genitalium. This follows from the unexpected finding that amplification and detection of the drug resistance marker is slightly more efficient when compared with amplification and detection of the wild-type (i.e., positive control) sequence. Accordingly, signal indicating detection of the drug resistance marker will emerge earlier than the signal indicating the presence of the wild-type sequence. The Ct value for the drug resistance marker will be a number smaller than the Ct value for the wild-type sequence, and so the ΔCt value will always be positive when processing nucleic acids of macrolide-resistant M. genitalium.

TABLE 1
Interpretation of Results

| ΔCt Processing Operation | Interpretation |
|---|---|
| Ct(HEX) − Ct(FAM) < 0 | SNP Negative (macrolide-sensitivity) |
| Ct(HEX) − Ct(FAM) > 0 | SNP Positive (macrolide-resistant) |

Of course, reversing the order of subtraction to determine ΔCt values (i.e., subtracting Ct(HEX) from Ct(FAM)) also can be used to establish macrolide sensitivity or resistance. Here a ΔCt greater than 0 would indicate macrolide-sensitivity, and a ΔCt value less than 0 would indicate macrolide resistance.

### Methods of Treatment and Changing Treatments

In some embodiments, the assays disclosed herein can be selected or ordered from a menu of testing options available to a healthcare professional caring for a human patient. For example, a physician may place an order using an electronic, paper, or other ordering system so that a sample obtained from the human patient will be subjected to the various steps needed to determine the presence or absence of macrolide-sensitive M. genitalium and/or of macrolide-resistant M. genitalium. In this regard, the individual placing the order or request can be said to “direct” or “have” certain steps performed for the purpose of making the determination regarding the presence or absence of the M. genitalium organism (e.g., the macrolide-resistant organism). For example, there can be a step for obtaining, or “having” obtained the sample to be used for testing, etc. Simply stated, the individual requesting an assay need not perform all of the procedural steps themselves. Of course, this might be considered relevant not only for initiating the sequence of events needed to obtain the molecular diagnostic result, but also relevant for automated systems, or data processing systems where data analysis is performed at a remote site.

In some embodiments, the molecular diagnostic assay is useful for detecting the presence of wildtype M. genitalium, and of determining the macrolide-resistance status of the organism, if present in the test sample. For example, a single test may combine detection of genetic markers for M. genitalium (e.g., the wildtype organism) and for macrolide-resistance. In a different embodiment, the assay for detecting macrolide-resistance can be performed on a test sample that previously was determined by independent testing to contain M. genitalium. This latter approach is sometimes referred to as a “reflex” test.

If it is determined that an M. genitalium-containing test sample obtained from a patient either includes or does not include macrolide-resistant M. genitalium, then a course of action can be implemented or changed to treat the patient for an improved outcome. If it is determined that the sample obtained from the patient includes macrolide-resistant M. genitalium, then the patient can be treated with a course of one or more antibiotics other than a macrolide antibiotic (e.g., azithromycin). For example, the treating healthcare professional may elect to prescribe, recommend, or treat with a fluoroquinolone antibiotic, or another agent effective against macrolide-resistant M. genitalium. Alternatively, if it is determined that the patient sample includes nucleic acids of M. genitalium, but does not include nucleic acids of macrolide-resistant M. genitalium, then a course of antibiotics other than fluoroquinolones may be prescribed or recommended. For example, a patient harboring an infection with M. genitalium that is not macrolide-resistant M. genitalium may be treated with a macrolide antibiotic (e.g., azithromycin) or another antibiotic effective against M. genitalium. Yet a different possibility is that a patient may have been treated with a course of fluoroquinolone antibiotics that will have been effective at controlling or eliminating an infection with macrolide-resistant M. genitalium. A subsequent test result indicating the absence of macrolide-resistant M. genitalium nucleic acid in a sample obtained following the initial treatment may guide the healthcare professional to change the treatment plan by discontinuing administration of the fluoroquinolone antibiotic (e.g., because it is no longer necessary).

### ILLUSTRATIVE EXAMPLES

The following Examples are provided to illustrate certain disclosed embodiments and are not to be construed as limiting the scope of the disclosure in any way.

Amplification reagents used in this procedure included: dNTPs at 0.2-0.8 mM each, a commercially available Hot Start Taq DNA polymerase (New England BioLabs; Ipswich, Mass.), MgCl₂, Cleavase® enzyme (Hologic, Inc.; San Diego, Calif.), MOPS and Tris buffers, non-acetylated BSA, dNTPs, and salts. The Afu FEN-1 endonuclease described in U.S. Pat. No. 9,096,893 can also be used in the invasive cleavage assay. Primers were supplied at a final concentration of 0.2-0.75 μM unless otherwise indicated.

Nucleic acid amplification products synthesized in the working Examples were detected by invasive cleavage reactions as amplification reactions were occurring. As noted elsewhere herein, wild-type positive control sequences were detected using an invasive cleavage reaction wherein one of the PCR primers served as the invasive probe to cleave a primary probe Amplicons indicating the presence of macrolide resistance markers employed an invasive probe that was not a PCR primer. For each primary probe there was a corresponding FRET cassette labeled with an interactive label pair in an energy transfer relationship, where fluorescence emission was quenched when both members of the label pair were attached to the FRET cassette. The wild-type sequence was detected using one FRET cassette, while the macrolide resistance markers (i.e., four different SNPs) were detected using a different FRET cassette. More particularly, all four of the different macrolide resistance markers were detected using the same FRET cassette that differed from the FRET cassette used for detecting the wild-type sequence. A positive signal for a given target was generally interpreted as indicating that a target sequence was present and amplified by a corresponding set of primers. Invasive cleavage structures that formed in the amplification reaction mixtures included an amplicon, an invasive probe (e.g., one of the PCR primers for cleaving the primary probe that detected the positive control amplicon; a dedicated invasive probe for cleaving the primary probes that detected macrolide resistance), and a primary probe. Following cleavage of a primary probe to release a 5′-flap, the cleaved 5′-flap interacted with its corresponding FRET cassette, which in turn was cleaved by the Cleavase® enzyme to release fluorophore and permit signal detection.

In certain highly preferred embodiments, real-time PCR with invasive cleavage detection was performed by combining template DNA with a solution that included primers, an invasive cleavage oligonucleotide, “SNP probes” (i.e., four primary probes that, in combination with the invasive cleavage oligonucleotide, detect the point mutations of the macrolide resistance markers in the 23S ribosomal nucleic acid), a positive control primary probe that detects a wild-type M. genitalium sequence amplified by the same primers that amplify the macrolide resistance markers, Taq DNA polymerase and Cleavase® enzymes, nucleotides, and buffer. The reaction mixture was preheated to 95° C. for 2 minutes, and a three-step PCR reaction was carried out for 40 cycles (95° C. for 15 seconds; 63° C. for 25 seconds; 72° C. for 40 seconds) using a commercially available real-time PCR instrument with fluorescent monitoring. Fluorescence signals were measured at the end of the incubation/extension step at 63° C. for each cycle.

In certain highly preferred embodiments, amplification and detection of wild-type and macrolide resistance markers were detected in multiplex reactions, where the different sequences within the same amplicon were detected using different fluorophores. For example, the wild-type positive control sequence was detected using a HEX fluorescent signal, and the macrolide resistance markers were detected using a FAM fluorescent signal in the multiplex reaction.

To be clear, macrolide resistance is indicated when either of two positions of the wild-type 23S ribosomal nucleic acid sequence of SEQ ID NO:23 are mutated. More specifically, position 508 of SEQ ID NO:23 corresponds to the position referenced herein as 2058 of region V in E. coli 23S ribosomal RNA. Position 509 of SEQ ID NO:23 corresponds to the position referenced herein as 2059 of region V in E. coli 23S ribosomal RNA. Macrolide resistance is indicated when the nucleotide A residue at position 508 of SEQ ID NO:23 is substituted by any of G, C or T. Alternatively, macrolide resistance is indicated when the nucleotide A residue at position 509 of SEQ ID NO:23 is substituted by G. The sequence of SEQ ID NO:24 particularly calls out the substitution of G in place of A at position 509 of the wild-type sequence given by SEQ ID NO:23.

Example 1 describes assessment of reverse primers used in the macrolide resistance assay. This procedure focused on the positive control feature of the assay, which employed the same forward and reverse primers that were used to amplify the macrolide resistance marker. Primer combinations were screened in amplification reaction mixtures that further included oligonucleotides needed for invasive cleavage detection reactions. Trials were performed using several concentrations of an input plasmid template harboring the wild-type M. genitialium 23S nucleic acid sequence. The most efficient primers yielded the earliest Ct (i.e., threshold cycle) values.

### Example 1

### Reverse Primer Screening

Real-time PCR reactions that amplified a segment of the M. genitialium 23S nucleic acid included oligonucleotides that permitted invasive cleavage detection of a wild-type sequence within the amplification product. These reactions were performed by combining 10 μl of a template DNA solution with 15 μl of a mixture containing oligonucleotide primers, a primary probe specific for a wild-type sequence within the amplification product, and a corresponding FRET cassette that hybridized the 5′-flap oligonucleotide cleaved from the wild-type primary probe to undergo a second cleavage reaction that separated a fluorophore from a quencher, thereby producing a detectable fluorescent signal. The wild-type primary probe was arranged so that the reverse primer functioned as an invasive probe. Also included in the PCR reactions were enzymes (Taq polymerase and Cleavase® enzyme), and pH buffer. The plasmid template used to prime the amplification reaction included the sequence of SEQ ID NO:23. Oligonucleotide reagents were as follows: the forward primer had the sequence of SEQ ID NO:1; reverse primers (tested separately) had the sequences of SEQ ID NO:2, SEQ ID NO:3, and SEQ ID NO:4; wild-type primary probe had the sequence of SEQ ID NO:10; and a FRET cassette used to indicate detection of the wild-type sequence had the sequence of SEQ ID NO:15. The FRET cassette included each of a FAM (fluorescein) fluorescent label moiety and a quencher moiety. Reaction mixtures were preheated at 95° C. for 2 minutes, and a three-step PCR reaction was carried out for 40 cycles (95° C. for 15 seconds, 63° C. for 25 seconds, and 72° C. for 40 seconds) using a commercially available real-time PCR instrument with fluorescent monitoring. Fluorescent signals detected at the FAM emission wavelength were measured at the end of the 63° C. incubation/extension step for each cycle. In this procedure, the FAM signal indicated detection of the wild-type M. genitialium 23s nucleic acid sequence. All reactions were carried out in triplicate.

The results presented in Table 2 indicated the reverse primer identified as SEQ ID NO:4 amplified the template nucleic acid most efficiently, as judged by producing the lowest Ct values. More particularly, reaction mixtures that included the reverse primer of SEQ ID NO:4 produced a predetermined level of amplification products more quickly than reaction mixtures that included the other reverse primers. For example, reaction mixtures primed with 10 copies of the template nucleic acid, and that included the reverse primer of SEQ ID NO:4 produced predetermined threshold levels of amplification products 5 and 10 cycles faster than reactions that included the other two primers. Use of the reverse primer of SEQ ID NO:4 facilitated detection over a wider dynamic range, possibly allowing detection down to a single copy of the starting template. The reverse primer of SEQ ID NO:4 was selected for subsequent studies.

TABLE 2
Assessing Amplification Efficiency by Measured FAM Ct Value

| Reverse | Input Template | Avg. Ct | Std. Dev. Ct | Avg. of RFU |
|---|---|---|---|---|
| Primer ID | (copies) | (cycles) | (cycles) | Range |
| | 1,000,000 | 23.12 | 0.54 | 40728 |
| Reverse 1 | 10,000 | 32.50 | 0.70 | 28315 |
| SEQ ID NO: 2 | 100 | 40.24 | 0.66 | 1059 |
| | 0 | N/A | N/A | 10296 |
| | 1,000,000 | 20.89 | 0.03 | 40636 |
| Reverse 2 | 10,000 | 29.53 | 0.50 | 35973 |
| SEQ ID NO: 3 | 100 | 35.65 | 0.13 | 7831 |
| | 0 | N/A | N/A | 9510 |
| | 1,000,000 | 16.61 | 0.41 | 40188 |
| Reverse 3 | 10,000 | 23.39 | 0.38 | 39192 |
| SEQ ID NO: 4 | 100 | 30.21 | 0.53 | 37113 |
| | 0 | N/A | N/A | 8728 |

Example 2 describes an alternative invasive probe that reduced background signal due to the presence of wild-type M. genitialium ribosomal nucleic acid. Plasmid DNA templates served as model wild-type and macrolide-resistant M. genitialium target nucleic acids. Background signal reduction advantageously improved results when testing samples containing mixed populations of macrolide-sensitive and macrolide-resistant M. genitialium.

### Example 2

### Invasive Probe Screening

Real-time PCR amplification with invasive cleavage detection of the amplified wild-type and macrolide resistance marker sequences was performed to assess influence of the invasive probe on background signal. In this procedure, the template nucleic acid harboring the macrolide resistance mutation included the sequence of SEQ ID NO:24. Wild-type template included about 720 bp of wild-type DNA sequence (SEQ ID NO:23) encoding the M. genitalium 23s rRNA. The invasive probe of SEQ ID NO:9 and the invasive probe of SEQ ID NO:8 were compared with each other for the ability to detect the macrolide resistance marker using a shared set of primary probes. Oligonucleotide reagents were as follows: the forward primer had the sequence of SEQ ID NO:1; the reverse primer had the sequence of SEQ ID NO:4; invasive probes (tested in independent reactions) had the sequence of either SEQ ID NO:8 or SEQ ID NO:9; primary probes (used in combination) had the sequences of SEQ ID NO:11, SEQ ID NO:12, SEQ ID NO:13, and SEQ ID NO:14; and a FRET cassette used to detect cleaved Flap Oligos of the primary probes had the sequence of SEQ ID NO:16. Again, reactions including one of the invasive probes undergoing comparison were primed using either plasmid DNA harboring the wild-type template or plasmid DNA harboring the macrolide resistance marker, each at 1×10⁶ input copies. Additional trials were primed using 1×10⁵ input copies of the template harboring the macrolide resistance marker. All trials were conducted in replicates of three. FAM fluorescence indicating amplification and detection of the macrolide resistance marker was monitored as a function of cycle number, as described under Example 1.

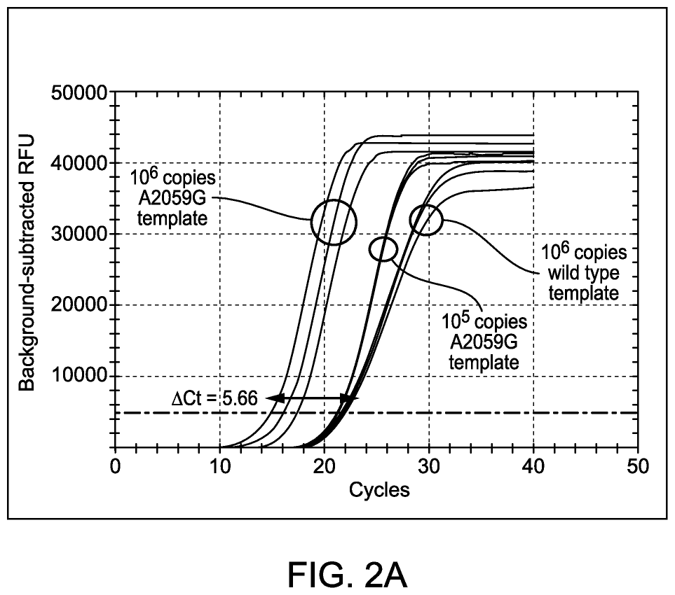

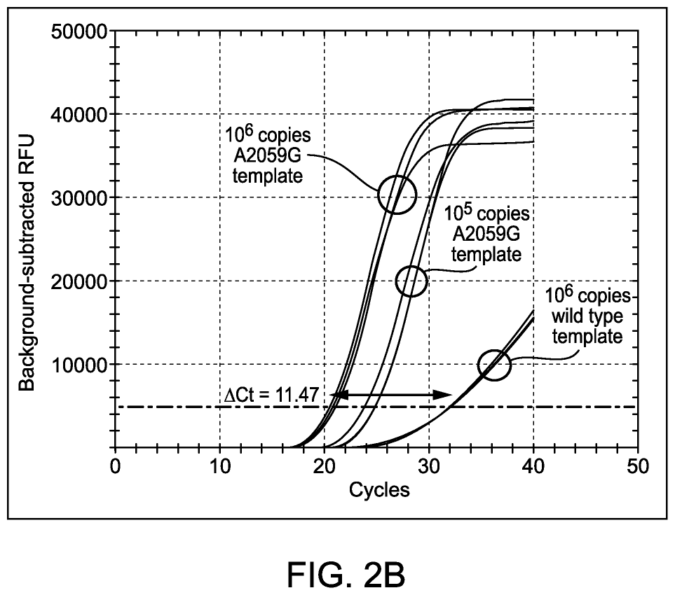

Results of the procedure are presented in FIGS. 2A-2B and Table 3. Trials conducted using 1×10⁶ copies of wild-type template and 1×10⁵ copies of template harboring the macrolide resistance marker were not clearly distinguished from each other in the real-time assay that included the invasive probe of SEQ ID NO:8 with monitoring of the FAM signal that indicated cleavage of the FRET cassette specific for macrolide resistance. Conversely, trials that included the invasive probe of SEQ ID NO:9 in place of the invasive probe of SEQ ID NO:8 showed clear distinctions. The run curve obtained using the wild-type template and the invasive probe of SEQ ID NO:9 dramatically shifted to a later emergence time compared to the run curve obtained using the wild-type template and the invasive probe of SEQ ID NO:8. The difference in Ct values measured for similar input copy levels of the macrolide resistance mutant and wild-type template advantageously increased from 5.66 cycles to 11.47 cycles as a result of the invasive probe substitution. Thus, using the invasive probe of SEQ ID NO:9 in place of the invasive probe of SEQ ID NO:8 advantageously reduced the wild-type background signal from about 50-fold less than the positive signal (i.e., 2^{5.66}) to nearly 3,000-fold less than the positive signal (i.e., 2^{11.47}). As well, the slope of the run curve obtained using the invasive probe of SEQ ID NO:9 was decreased compared to the trial conducted using the invasive probe of SEQ ID NO:8. This advantageously allows flexibility in setting background cutoff parameters. The invasive probe of SEQ ID NO:9 was selected for use in subsequent procedures. Entries in Table 3 given as “N/A” indicate that no amplification was detected.

TABLE 3
Invasive Probe Reduces Background Signal

| | | | |
|---|---|---|---|
| Reaction Composition | Avg. Ct | St. Dev. Ct | Avg. RFU Range |

Invasive Probe: SEQ ID NO: 8

| | | | |
|---|---|---|---|
| 1 × 10⁶ copies wild-type template | 21.58 | 0.26 | 38505 |
| 1 × 10⁶ copies A2059G template | 15.92 | 1.15 | 42818 |
| 1 × 10⁵ copies A2059G template | 21.10 | 0.05 | 40939 |
| No Template Control | 73.59 | 17.39 | 1493 |

Invasive Probe: SEQ ID NO: 9

| | | | |
|---|---|---|---|
| 1 × 10⁶ copies wild-type template | 32.04 | 0.07 | 15961 |
| 1 × 10⁶ copies A2059G template | 20.57 | 0.33 | 39351 |
| 1 × 10⁵ copies A2059G template | 24.28 | 0.66 | 39765 |
| No Template Control | N/A | N/A | N/A |

Use of the invasive probe of SEQ ID NO:9 advantageously reduced the background signal, but undesirably reduced assay sensitivity. Reduced sensitivity was reflected by increases in the average Ct values. For example, the Ct value measured for trials conducted using 1×10⁶ input copies of the mutant template harboring the macrolide resistance marker increased from 15.92 cycles to 20.57 cycles as a result of substituting the invasive probe of SEQ ID NO:9 in place of the invasive probe of SEQ ID NO:8 (see Table 3). Although both assays were fully functional, there was a desire to improve assay sensitivity even further by modifying the reverse primer.

Example 3 describes refinement of the reverse primer design to improve assay sensitivity. This was accomplished by modifying the sequence of the reverse primer so that the Tm for hybridization to the complementary strand of the amplification product more closely matched the Tm for hybridization of the forward primer to the complementary strand of the amplification product.

### Example 3

### Improved Reverse Primer Enhances Assay Sensitivity

Modified versions of each of the three reverse primers from Example 1 were prepared using oligonucleotide chemical synthetic procedures familiar those having an ordinary level of skill in the art. Sequences of the modified primers included the sequences of the corresponding reverse primers from Example 1 appended to additional sequences at their 5′-ends. The sequence of the Reverse 1′ reverse primer (SEQ ID NO:5) included the sequence of SEQ ID NO:2 appended to, at the 5′-end, three nucleotides complementary to the M. genitialium 23S rRNA target, and an additional six nucleotides that are not complementary to the rRNA. The sequence of the Reverse 2′ reverse primer of SEQ ID NO:6 included the sequence of SEQ ID NO:3 appended to, at the 5′-end, two nucleotides complementary to the M. genitialium 23s rRNA target, and an additional six nucleotides that are not complementary to the rRNA. The sequence of the Reverse 3′ reverse primer of SEQ ID NO:7 included the sequence of SEQ ID NO:4 appended to, at the 5′-end, six nucleotides that are not complementary to the M. genitialium 23s rRNA target. All three of the alternative primers, as well as the comparator primer from Example 1 (SEQ ID NO:4), were separately used for amplifying and detecting nucleic acid sequences conferring macrolide resistance in reaction mixtures that included: the forward primer of SEQ ID NO:1; the invasive probe of SEQ ID NO:9, primary probes (used in combination) having the sequences of SEQ ID NO:11, SEQ ID NO:12, SEQ ID NO:13, and SEQ ID NO:14; and a FRET cassette had the sequence of SEQ ID NO:16. All reactions were primed with 1×10⁶ copies of a plasmid template harboring the A2058C macrolide resistance mutation. Synthesis of amplification products was monitored as a function of reaction cycle number, as described under Example 1.

Results of the procedure are summarized in Table 4. All the alternative reverse primers performed well in the real-time assays, and so can be used for detecting drug-resistant M. genitialium. Each of reverse primers identified as Reverse 1′ (SEQ ID NO:5), Reverse 2′ (SEQ ID NO:6), and Reverse 3′ (SEQ ID NO:7) advantageously led to lower average Ct values (i.e., more rapid times of emergence) relative to the comparator Reverse 3 reverse primer (SEQ ID NO:4). The Reverse 3′ primer was selected for use in subsequent procedures.

TABLE 4
Comparison of Alternative Reverse Primers

| | | Avg. Ct | Std. Dev. of Ct |
|---|---|---|---|
| Primer | Reverse Primer ID | (cycles) | (cycles) |
| Comparator | SEQ ID NO: 4 | 22.12 | 0.36 |
| Reverse 1′ | SEQ ID NO: 5 | 13.55 | 0.36 |
| Reverse 2′ | SEQ ID NO: 6 | 14.16 | 0.13 |
| Reverse 3′ | SEQ ID NO: 7 | 13.51 | 0.32 |

Example 4 describes integration of a target isolation step into the assay workflow. It is to be understood that target nucleic acids can be isolated by immobilization to a solid support in a variety of ways. This can involve sequence-specific hybridization of the target nucleic acid to an immobilized oligonucleotide. Optionally, there can be a third molecule (e.g., a “target capture oligonucleotide”) that bridges the immobilized oligonucleotide and the target nucleic acid. The M. genitialium M30 strain that included wild-type 23S rRNA sequences (i.e., no macrolide resistance mutations or “markers”) was used to demonstrate the target capture step. Success of the procedure indicated that templates harboring the macrolide resistance marker also could be captured and processed in the same manner The target capture oligonucleotides (TCOs) used in the procedure are merely illustrative, and alternative TCOs can be substituted.

### Example 4

### Integrated Target Capture

In vitro transcripts (IVTs) of the M. genitialium 23S rRNA were enriched by target capture preliminary to PCR amplification with invasive cleavage detection. Target capture probes used in the procedure had 5′ target binding regions of SEQ ID Nos:17-19, and further included 3′ immobilized probe binding regions, where the 3′ immobilized probe binding regions included poly(dA) tails 30 nucleotides in length. Target binding sequences (e.g., SEQ ID Nos:17-19) were synthesized using nucleotide analogs having 2′-methoxy (2′-OMe) modifications on the pentose. The target binding region of the capture probe was designed to bind to a region of the target nucleic acid that was distinct from the regions bound by primers, the invasive probe used for detecting the macrolide resistance marker, and the primary probes. The immobilized probe binding regions facilitated hybridization to an immobilized probe disposed on the solid support. In this example, the immobilized probe included an oligo(dT) sequence. The full target capture oligonucleotide sequences were given by SEQ ID Nos:20-22. The solid support of this target capture step can be a Sera-Mag™ MG-CM Carboxylate Modified (Seradyn, Inc.; Indianapolis, Ind.; Cat. No. 24152105-050450), 1 micron, super-paramagnetic particle having a covalently bound oligo(dT)₁₄ which was able to bind to the poly(dA) tail of the capture probe under hybridization conditions. Similar magnetic particles are disclosed by Sutor, “Process for Preparing Magnetically Responsive Microparticles,” U.S. Pat. No. 5,648,124. To draw the particles out of suspension and immobilize them along the inner wall of the sample tubes, the tubes were transferred to a magnetic separation rack essentially as disclosed by Acosta et al. in U.S. Pat. No. 6,254,826. While the particles were immobilized, fluid was aspirated from the tubes and the tubes were washed with a wash buffer. The wash step optionally can be repeated before adding each of an amplification reagent that included nucleotides and cofactors, and an enzyme reagent that included a reverse transcriptase, Taq DNA polymerase, and Cleavase® enzyme.

Lysates of M. genitialium bacterial strain M30 were incubated with a target capture oligo (TCO) specific for the 23s rRNA and the above-described solid support having oligo(dT)₁₄ immobilized thereon for 30 minutes at 62° C., and then at room temperature for 20 minutes. Amounts of lysate used in the procedure corresponded to 100, 10, and 1 cfu/ml. Three different TCOs were used independently of one another in the procedure. Complexes including a TCO and 23s rRNA were purified by magnetic particle separation, washing, and elution into water using a commercially available robotic magnetic particle processor. Next, 10 μl of the eluted rRNA template was combined with a 15 μl reaction mixture that included primers, an invasive probe specific for the macrolide resistance marker, primary probes specific for wild-type and macrolide resistance markers, and corresponding FRET cassettes. Also included were enzymes (Taq DNA polymerase, Cleavase® enzyme, and a reverse transcriptase) and a buffered solution that included nucleotides and cofactors used in the real-time PCR reaction with invasive cleavage detection of amplification products. Reaction mixtures were heated at 50° C. for 5 minutes to perform the reverse transcription step. Mixtures were then heated to 95° C. for 2 minutes to inactivate the reverse transcriptase and preheat the cycling reaction. Next, a three-step PCR reaction was carried out for 40 cycles (95° C. for 15 seconds, 63° C. for 25 seconds, and 72° C. for 40 seconds) using a real-time instrument with fluorescent monitoring. Fluorescence values of FAM and/or HEX were measured at the end of the incubation/extension step at 63° C. for each cycle. A threshold-based run curve analysis familiar to those having an ordinary level of skill in the art was used to determine Ct values for the different reactions.

The results of these procedures, presented in Table 5, confirmed that a target capture step could be integrated into the assay workflow.

TABLE 5
Detection of M. genitalium Nucleic Acid Sequences Following
Target Enrichment

| | M. gen | | | |
|---|---|---|---|---|
| (Target Capture | Lysate | | M. gen IVT | |
| Oligo) | (CFU/ml) | Ct Value | RNA Copies | Ct Value |
| SEQ ID NO: 17 | 100 | 25.97 | 1,000,000 | 26.52 |
| (SEQ ID NO: 20) | 10 | 29.94 | 100,000 | 30.60 |
| | 1 | 32.53 | 10,000 | 33.16 |
| | 0.1 | 33.43 | N/A | N/A |
| SEQ ID NO: 18 | 100 | 25.97 | 1,000,000 | 26.52 |
| (SEQ ID NO: 21) | 10 | 29.94 | 100,000 | 30.60 |
| | 1 | 32.53 | 10,000 | 33.16 |
| | 0.1 | 33.43 | N/A | N/A |
| SEQ ID NO: 19 | 100 | 25.97 | 1,000,000 | 26.52 |
| (SEQ ID NO: 22) | 10 | 29.94 | 100,000 | 30.60 |
| | 1 | 32.53 | 10,000 | 33.16 |
| | 0.1 | 33.43 | N/A | N/A |

Example 5 describes use of the disclosed technique for detecting both M. genitialium wild-type positive control target sequence, and M. genitialium macrolide resistance markers. An alternative embodiment that detects macrolide resistance markers without also detecting the wild-type sequence can omit the wild-type primary probe (e.g., SEQ ID NO:10) and the corresponding signal-generating FRET cassette (e.g., SEQ ID NO:15). In accordance with yet another alternative embodiment, detecting additional variants at nucleotide positions indicating macrolide resistance (i.e., positions 2058 or 2059) can involve supplementing the below described reaction mixture with one or more additional primary probes, and optionally one or more invasive probes. This can be done, for example, to enhance assay functionality to include detection of at A to C base change at position 2059. The same approach can be used to enhance functionality of any other reaction mixture disclosed herein.

### Example 5

### Testing In Vitro-Cultured Clinical Strains of M. genitialium for Macrolide resistance

M. genitalium clinical strains developed from the culture of clinical isolates, and characterized for azithromycin (AZM) sensitivity, were analyzed using the disclosed macrolide resistance assay. DNA isolated from the samples was used to prime real-time PCR with invasive cleavage detection of amplification products. Forward and reverse primers used in the reaction were SEQ ID NO:1 and SEQ ID NO:7, respectively. The primary probe for detecting the wild-type sequence was SEQ ID NO:10. Primary probes for detecting macrolide resistance markers had the sequences of: SEQ ID NO:11, SEQ ID NO:12, SEQ ID NO:13, and SEQ ID NO:14. The invasive probe for detecting macrolide resistance markers had the sequence of SEQ ID NO:9. The reverse primer functioned as an invasive probe specific for the wild-type primary probe. A FRET cassette for detecting wild-type sequences harbored a HEX label and had the sequence of SEQ ID NO:15. A FRET cassette for detecting macrolide resistance markers harbored a FAM label and had the sequence of SEQ ID NO:16.

Results of the procedure, presented in Table 6, confirmed perfect agreement between the disclosed real-time assay result and independent testing for macrolide resistance using standard microbiological culture techniques.

TABLE 6
Clinical Testing Results

| | | | | | MgenR Real- | |
|---|---|---|---|---|---|---|
| | AZM | HEX | FAM | | Time Assay | |
| Strain ID | Type* | Ct | Ct | ΔCt | Result | Agreement |
| Mega-216 | 2 | 18.23 | 17.51 | 0.72 | SNP Positive | Concordant |
| Mega-1272 | 1 | 16.48 | 14.49 | 1.99 | SNP Positive | Concordant |
| Mega-1256 | 3 | 15.60 | 14.61 | 0.99 | SNP Positive | Concordant |
| Mega-1082 | 3 | 16.65 | 15.52 | 1.13 | SNP Positive | Concordant |
| 100080-1 | 3 | 20.67 | 20.27 | 0.40 | SNP Positive | Concordant |
| Mega-601 | 0 | 16.34 | 20.55 | −4.21 | SNP Negative | Concordant |
| Mega-1331 | 0 | 15.26 | 19.67 | −4.41 | SNP Negative | Concordant |
| Mega-1402 | 0 | 18.68 | 22.37 | −3.69 | SNP Negative | Concordant |
| Sea-1 | — | 16.73 | 20.02 | −3.29 | SNP Negative | Concordant |
| Sea-2 | — | 15.64 | 19.93 | −4.29 | SNP Negative | Concordant |

*AZM 0 = Azithromycin-sensitive (wild-type)
AZM 1, 2, 3 = Azithromycin-resistant

All of the compositions, kits, and methods disclosed and claimed herein can be made and executed without undue experimentation in light of the present disclosure. While the disclosure describes preferred embodiments, it will be apparent to those of skill in the art that variations may be applied without departing from the spirit and scope of the disclosure. All such variations and equivalents apparent to those skilled in the art, whether now existing or later developed, are deemed to be within the spirit and scope of the disclosure.

All patents, patent applications, and publications mentioned in the specification are indicative of the levels of those of ordinary skill in the art to which the disclosure pertains. All patents, patent applications, and publications are herein incorporated by reference in their entirety for all purposes and to the same extent as if each individual publication was specifically and individually indicated to be incorporated by reference in its entirety for any and all purposes.

What is claimed is:

1. A method of determining the presence or absence of a nucleic acid target sequence in a test sample, the method comprising the steps of:
(a) obtaining nucleic acid from the test sample;
(b) performing an in vitro nucleic acid amplification reaction using a pair of primers and nucleic acid obtained in step (a) as templates to produce an amplification product comprising first and second nucleic acid strands that are complementary to each other,
wherein the first nucleic acid strand comprises a positive control sequence, and
wherein the second nucleic acid strand may comprise the nucleic acid target sequence;
(c) detecting, as the in vitro nucleic acid amplification reaction is taking place, the positive control sequence in the first nucleic acid strand and any of the nucleic acid target sequence that may be present in the second nucleic acid strand to determine Ct values for each of the positive control sequence and the nucleic acid target sequence; and
(d) comparing the determined Ct values to establish the presence or absence of the nucleic acid target sequence in the test sample.

2. The method of claim 1, wherein step (c) comprises detecting with invasive cleavage reactions.

3. The method of claim 2, wherein Ct values determined for the positive control sequence and the nucleic acid target sequence are not identical when both the positive control sequence and the nucleic acid target sequence are both present in the amplification product produced in step (b).

4. A method of determining the macrolide resistance status of M. genitalium in a test sample, the method comprising the steps of:
(a) obtaining nucleic acid from M. genitalium of the test sample;
(b) performing an in vitro nucleic acid amplification reaction using nucleic acid obtained in step (a) as templates to produce an amplification product comprising a segment of M. genitalium 23S ribosomal nucleic acid,
wherein the segment comprises two adjacent nucleotide positions, corresponding to positions 2058 and 2059 of region V in E. coli 23S rRNA, that distinguish macrolide-sensitive and macrolide-resistant M. genitalium, and
wherein the segment further comprises a wild-type sequence of M. genitalium 23S ribosomal nucleic acid;
(c) detecting in the amplification product, as the in vitro nucleic acid amplification reaction of step (b) is occurring, the wild-type sequence, and any of a macrolide resistance marker that may be present at either of said two adjacent nucleotide positions to determine Ct values for each of the wild-type sequence and the macrolide resistance marker; and
(d) comparing the determined Ct values to establish the presence or absence of the macrolide resistance marker in the amplification product, thereby determining the macrolide resistance status of M. genitalium in the test sample.

5. The method of claim 4, wherein the amplification product produced in the in vitro nucleic acid amplification reaction of step (b) comprises a double-stranded DNA.

6. The method of claim 5, wherein step (c) comprises detecting the wild-type sequence and the macrolide resistance marker on different strands of the double-stranded DNA.

7. The method of claim 5, wherein the in vitro nucleic acid amplification reaction of step (b) comprises a flap endonuclease (FEN) enzyme, and wherein step (c) comprises detecting with a plurality of invasive cleavage reactions.

8. The method of claim 7, wherein the in vitro nucleic acid amplification reaction is a PCR reaction employing first and second primers oriented opposite to each other, and wherein one of the primers is an invasive probe that promotes cleavage of a first primary probe to release a first 5′-flap oligonucleotide in the presence of the FEN enzyme.

9. The method of claim 8, wherein the first primary probe is specific for the wild-type sequence, and is cleaved by the FEN enzyme if hybridized to any of the amplification product that comprises the wild-type sequence.

10. The method of claim 4, wherein the macrolide resistance marker is either A2058C, A2058T, or A2058G.

11. The method of claim 4, wherein the macrolide resistance marker is A2059G.

12. The method of claim 5, wherein step (c) comprises detecting with a plurality of invasive cleavage reactions.

13. The method of claim 12, wherein the plurality of invasive cleavage reactions distinguishes the wild-type sequence from the macrolide resistance marker, but does not distinguish any of A2059G, A2058C, A2058T or A2058G from each other.

14. The method of claim 5, wherein a set of four primary probes is used to detect the macrolide resistance marker at either of said two adjacent nucleotide positions in one strand of the double-stranded DNA, and wherein each probe among the set shares the same 5′-flap sequence.

15. The method of claim 5, wherein a set of four primary probes is used to detect the macrolide resistance marker at either of said two adjacent nucleotide positions in one strand of the double-stranded DNA, and wherein step (c) comprises detecting with a single invasive probe that cleaves a 5′-flap from any of the four primary probes among the set in the presence of a complementary DNA strand comprising any of A2059G, A2058C, A2058T and A2058G.

16. The method of claim 13, wherein cleavage of a single FRET cassette separates a fluorophore and a quencher following hybridization of the single FRET cassette and a 5′-flap cleaved from any primary probe among the set of four primary probes.

17. The method of claim 4, wherein step (d) comprises calculating a difference between the Ct values.

18. The method of claim 4, wherein step (d) comprises calculating a difference between the Ct values, and then determining whether the difference is greater than or less than 0 cycles.

19. The method of claim 4, wherein the test sample comprises a clinical swab sample obtained from a patient.

20. The method of claim 4, wherein step (a) comprises obtaining RNA from M. genitalium of the test sample, and wherein step (b) comprises performing the in vitro nucleic acid amplification reaction using the RNA obtained in step (a) as templates.

21. The method of claim 4, wherein the test sample comprises a mixture of macrolide-resistant M. genitalium and macrolide-sensitive M. genitalium.

22. The method of claim 7, wherein the test sample is known to comprise M. genitalium prior to performing step (b), and wherein step (c) comprises detecting with two different FRET cassettes, each FRET cassette being labeled with a different fluorophore.

23. The method of claim 4, wherein step (a) comprises obtaining nucleic acids by hybridization capture onto a solid support displaying immobilized oligonucleotides.

24. An oligonucleotide composition, comprising:
(a) a first primer complementary to a sequence of M. genitalium 23S rRNA or a DNA equivalent strand downstream of position 2059 of corresponding region V in E. coli 23S rRNA, and a second primer complementary to an extension product of the first primer using M. genitalium 23S rRNA or the DNA equivalent strand as a template, the second primer being complementary to a sequence of M. genitalium 23S ribosomal DNA upstream of position 2058 of corresponding region V in E. coli 23S rRNA;
(b) a primary probe comprising a wild-type target-binding sequence attached to an upstream 5′-flap sequence,
wherein the wild-type target-binding sequence is complementary to a wild-type sequence of M. genitalium 23S rRNA downstream of the first primer with a 1-2 base overlap at the 5′-end of the wild-type target-binding sequence when the primary probe and the first primer are hybridized to the same strand of M. genitalium 23S rRNA or the DNA equivalent strand;
(c) a set of four primary probes, each probe of the set being specific for a different single nucleotide polymorphism (SNP) in M. genitalium 23S ribosomal DNA, at positions corresponding to positions 2058 and 2059 of region V in E. coli 23S rRNA, that distinguishes macrolide-sensitive and macrolide-resistant M. genitalium,
wherein each of the four primary probes is specific for one of A2058C, A2058T, A2058G, and A2059G, and
wherein each primary probe among the set is attached to an upstream 5′-flap sequence different from the upstream 5′-flap sequence of the primary probe comprising the wild-type target-binding sequence;
(d) an invasive probe that promotes flap endonuclease (FEN) enzyme-mediated cleavage of a complex comprising the invasive probe, any of the set of four primary probes, and an M. genitalium 23S ribosomal DNA sequence from a macrolide-resistant M. genitalium but not macrolide-sensitive M. genitalium; and
(e) two FRET cassettes, one FRET cassette being specific for any cleaved 5′-flap released from the primary probe comprising the wild-type target-binding sequence, and the other FRET cassette being specific for any cleaved 5′-flap released from any of the set of four primary probes.

25. The oligonucleotide composition of claim 24, wherein the first primer comprises the sequence of SEQ ID NO:7.

26. The oligonucleotide composition of claim 24, wherein the second primer comprises the sequence of SEQ ID NO:1.

27. The oligonucleotide composition of claim 24, the primary probe comprising the wild-type target-binding sequence comprises the target-binding sequence of SEQ ID NO:10.

28. The oligonucleotide composition of claim 24, wherein the set of four primary probes comprises a probe of the sequence SEQ ID NO:11.

29. The oligonucleotide composition of claim 24, wherein the set of four primary probes comprises a probe of the sequence SEQ ID NO:12.

30. The oligonucleotide composition of claim 24, wherein the set of four primary probes comprises a probe of the sequence SEQ ID NO:13.

31. The oligonucleotide composition of claim 24, wherein the set of four primary probes comprises a probe of the sequence SEQ ID NO:14.

32. The oligonucleotide composition of claim 24, wherein the invasive probe comprises a sequence selected from the group consisting of SEQ ID NO:8 and SEQ ID NO:9.

33. The oligonucleotide composition of claim 24, wherein the primary probe comprising the wild-type target-binding sequence and each probe among the set of four primary probes are complementary to opposite strands of M. genitalium 23S ribosomal DNA.

34. A reaction mixture, comprising:
(a) an oligonucleotide composition in accordance with claim B10;
(b) a DNA polymerase;
(c) a FEN enzyme;
(d) dNTPs; and
(e) a 23S M. genitalium ribosomal nucleic acid.


SEQUENCE LISTING (24 sequences)

SEQ ID NO: 1 - DNA, 25 bp, Mycoplasma genitalium
  misc_feature (1)..(25): Forward primer
ctcggctatagactcggtgaaatcc

SEQ ID NO: 2 - DNA, 14 bp, Mycoplasma genitalium
  misc_feature (1)..(14): Reverse primer
tacatggtggtgtt

SEQ ID NO: 3 - DNA, 15 bp, Mycoplasma genitalium
  misc_feature (1)..(15): Reverse primer
ctacatggtggtgtt

SEQ ID NO: 4 - DNA, 17 bp, Mycoplasma genitalium
  misc_feature (1)..(17): Reverse primer
ctctacatggtggtgtt

SEQ ID NO: 5 - DNA, 23 bp, Artificial Sequence
  Reverse primer based on Mycoplasma genitalium
  misc_feature (1)..(6): 5′ sequence not complementary to ribosomal nucleic acid of Mycoplasma genitalium
  misc_feature (7)..(23): Sequence complementary to ribosomal nucleic acid of Mycoplasma genitalium
ccacgtctctacatggtggtgtt

SEQ ID NO: 6 - DNA, 23 bp, Artificial Sequence
  Reverse primer based on Mycoplasma genitalium
  misc_feature (1)..(6): 5′ sequence not complementary to ribosomal nucleic acid of Mycoplasma genitalium
  misc_feature (7)..(23): Sequence complementary to ribosomal nucleic acid of  Mycoplasma genitalium
cctattctctacatggtggtgtt

SEQ ID NO: 7 - DNA, 23 bp, Artificial Sequence
  Reverse primer based on Mycoplasma genitalium
  misc_feature (1)..(6): 5′ sequence not complementary to nucleic acid of Mycoplasma genitalium
  misc_feature (7)..(23): Sequence complementary to ribosomal nucleic acid of Mycoplasma genitalium
ggtgttctctacatggtggtgtt

SEQ ID NO: 8 - DNA, 16 bp, Mycoplasma genitalium
  misc_feature (1)..(16): Invasive probe
ggcgcaacgggacggg

SEQ ID NO: 9 - DNA, 17 bp, Mycoplasma genitalium
  misc_feature (1)..(17): Invasive probe
ggcgcaacgggacgggt

SEQ ID NO: 10 - DNA, 34 bp, Artificial Sequence
  Primary probe based on wild-type Mycoplasma genitalium
  misc_feature (1)..(12): 5′-flap equence not complementary to ribosomal nucleic acid of Mycoplasma genitalium
  misc_feature (13)..(34): Sequence complementary to ribosomal nucleic acid of Mycoplasma genitalium
acggacgcggagtttgatcaatattaagctacag

SEQ ID NO: 11 - DNA, 26 bp, Artificial Sequence
  Primary probe based on macrolide-resistant Mycoplasma genitalium
  misc_feature (1)..(10): 5′-flap sequence not complementary to ribosomal nucleic acid of Mycoplasma genitalium
  misc_feature (11)..(26): Sequence complementary to ribosomal nucleic acid of Mycoplasma genitalium
cgcgccgaggagagaccccgtgaagc

SEQ ID NO: 12 - DNA, 26 bp, Artificial Sequence
  Primary probe based on macrolide-resistant Mycoplasma genitalium
  misc_feature (1)..(10): 5′-flap sequence not complementary to ribosomal nucleic acid of Mycoplasma genitalium
  misc_feature (11)..(26): Sequence complementary to ribosomal nucleic acid of Mycoplasma genitalium
cgcgccgagggaagaccccgtgaagc

SEQ ID NO: 13 - DNA, 26 bp, Artificial Sequence
  Primary probe based on macrolide-resistant Mycoplasma genitalium
  misc_feature (1)..(10): 5′-flap sequence not complementary to ribosomal nucleic acid of Mycoplasma genitalium
  misc_feature (11)..(26): Sequence complementary to ribosomal nucleic acid of Mycoplasma genitalium
cgcgccgaggcaagaccccgtgaagc

SEQ ID NO: 14 - DNA, 26 bp, Artificial Sequence
  Primary probe based on macrolide-resistant Mycoplasma genitalium
  misc_feature (1)..(10): 5′-flap sequence not complementary to ribosomal nucleic acid of Mycoplasma genitalium
  misc_feature (11)..(26): Sequence complementary to ribosomal nucleic acid of Mycoplasma genitalium
cgcgccgaggtaagaccccgtgaagc

SEQ ID NO: 15 - DNA, 35 bp, Artificial Sequence
  FRET cassette to detect wild-type ribosomal sequences in Mycoplasma genitalium
tctagccggttttccggctgagactccgcgtccgt

SEQ ID NO: 16 - DNA, 33 bp, Artificial Sequence
  FRET cassette to detect macrolide resistance sequences in Mycoplasma genitalium
tctagccggttttccggctgagacctcggcgcg

SEQ ID NO: 17 - RNA, 24 nt, Mycoplasma genitalium
  misc_feature (1)..(24): Target-binding sequence of target capture oligonucleotide
cccucguucuucacugcggcuuac

SEQ ID NO: 18 - RNA, 24 nt, Mycoplasma genitalium
  misc_feature (1)..(24): Target-binding sequence of target capture oligonucleotide
cauauacaucaccuuacgguuugg

SEQ ID NO: 19 - RNA, 21 nt, Mycoplasma genitalium
  misc_feature (1)..(21): Target-binding sequence of target capture oligonucleotide
ccuuugugcgccuccguuacc

SEQ ID NO: 20 - DNA, 57 bp, Artificial Sequence
  Artificial sequence based on Mycoplasma genitalium
  misc_feature (1)..(57): Target capture oligonucleotide
cccucguucuucacugcggcuuactttaaaaaaaaaaaaaaaaaaaaaaaaaaaaaa

SEQ ID NO: 21 - DNA, 57 bp, Artificial Sequence
  Artificial sequence based on Mycoplasma genitalium
  misc_feature (1)..(57): Target capture oligonucleotide
cauauacaucaccuuacgguuuggtttaaaaaaaaaaaaaaaaaaaaaaaaaaaaaa

SEQ ID NO: 22 - DNA, 54 bp, Artificial Sequence
  Artificial sequence based on Mycoplasma genitalium
  misc_feature (1)..(54): Target capture oligonucleotide
ccuuugugcgccuccguuacctttaaaaaaaaaaaaaaaaaaaaaaaaaaaaaa

SEQ ID NO: 23 - DNA, 691 bp, Mycoplasma genitalium
  misc_feature (1)..(691): Wild-type Mycoplasma genitalium 23S ribosomal nucleic acid sequence
tctatgcttcacggaattcggcaattaatcatgctttcaagaaaagctgctagggctaatttaactgtaaccagtaccgagaacgaacacacgtagtcaaggagaggatcctaaggttagcgagtgaactatagccaaggaactctgcaaattaaccccgtaagttagcgagaaggggtgcttatctaaaagtaagccgcagtgaagaacgaggggggactgtttaactaaaacacaactctatgccaaaccgtaaggtgatgtatatggggtgacacctgcccagtgctggaaggttaaagaaggaggttagcaatttattgcaaagcttttaactgaagccccagtgaacggcggccgtaactataacggtcctaaggtagcgaaattcctagtcgggtaaattccgtcccgcttgaatggtgtaaccatctcttgactgtctcggctatagactcggtgaaatccaggtacgggtgaagacacccgttaggcgcaacgggacggaaagaccccgtgaagctttactgtagcttaatattgatcaaaacaccaccatgtagagaataggtaggagcaattgatgcaagttcgcaaggatttgttgatgtgaaatgtggaatactacccttggttatgttttgttctaattggtaactgtgatccaggcggccgctgctacactggatcc

SEQ ID NO: 24 - DNA, 691 bp, Mycoplasma genitalium
  misc_feature (1)..(691): Macrolide-resistant Mycoplasma genitalium 23S ribosomal nucleic acid sequence
  misc_feature (509)..(509): The G nucleotide at position 509 is characteristic of macrolide resistance
tctatgcttcacggaattcggcaattaatcatgctttcaagaaaagctgctagggctaatttaactgtaaccagtaccgagaacgaacacacgtagtcaaggagaggatcctaaggttagcgagtgaactatagccaaggaactctgcaaattaaccccgtaagttagcgagaaggggtgcttatctaaaagtaagccgcagtgaagaacgaggggggactgtttaactaaaacacaactctatgccaaaccgtaaggtgatgtatatggggtgacacctgcccagtgctggaaggttaaagaaggaggttagcaatttattgcaaagcttttaactgaagccccagtgaacggcggccgtaactataacggtcctaaggtagcgaaattcctagtcgggtaaattccgtcccgcttgaatggtgtaaccatctcttgactgtctcggctatagactcggtgaaatccaggtacgggtgaagacacccgttaggcgcaacgggacggagagaccccgtgaagctttactgtagcttaatattgatcaaaacaccaccatgtagagaataggtaggagcaattgatgcaagttcgcaaggatttgttgatgtgaaatgtggaatactacccttggttatgttttgttctaattggtaactgtgatccaggcggccgctgctacactggatcc